(19) United States
(12) Patent Application Publication
(10) Pub. No.: US 2015/0374210 A1
(43) Pub. Date: Dec. 31, 2015

(54) PHOTOMETRIC STEREO ENDOSCOPY

(71) Applicant: MASSACHUSETTS INSTITUTE OF TECHNOLOGY, Cambridge, MA (US)

(72) Inventors: Nicholas J. Durr, Cambridge, MA (US); Vicente Jose Parot, Somerville, MA (US); Daryl Lim, Boston, MA (US); German Gonzalez Serrano, Cambridge, MA (US)

(21) Appl. No.: 14/758,755

(22) PCT Filed: Mar. 13, 2014
(86) PCT No.: PCT/US2014/026881

§ 371 Date: Jun. 30, 2015

Related U.S. Application Data
(60) Provisional application No. 61/780,190, filed on Mar. 13, 2013.

(51) Int. Cl. A61B 1/00; A61B 1/04; A61B 1/31; A61B 1/07

(52) U.S. Cl. CPC A61B 1/00193; A61B 1/00009; A61B 1/07; A61B 1/041; A61B 1/31

(57) ABSTRACT

The present invention relates to systems and methods for photometric endoscope imaging. The methods can further include chromoendoscopy and computer aided detection procedures for the imaging of body lumens and cavities.

### CROSS-REFERENCE TO RELATED APPLICATIONS

This application claims the benefit of priority to U.S. Provisional Application Ser. No. 61/780,190, titled “Photometric Stereo Endoscopy,” and filed Mar. 13, 2013, the content of which is hereby incorporated by reference in its entirety.

### BACKGROUND OF THE DISCLOSURE

The present disclosure relates to the field of photometric endoscopic imaging more particularly as applied in the context of endoscopy. The present disclosure also relates to the fields of endoscopic screening, chromoendoscopy, and computer aided detection (CAD).

While, conventional video endoscopy has revolutionized the evaluation of the gastrointestinal tract and other body lumens and cavities, it is limited by its inability to extract significant topographical information. For many applications of endoscopy, the observation of tissue surface morphology is critically important for effective screening. Consider, for example, screening for colorectal cancer, where lesions are characterized not only by color differences, but can also often be identified by their protrusion above or below the surrounding mucosa. In computed tomography colonography, for instance, the shape of the colon mucosa alone is sufficient to identify lesions. Colorectal cancer is the second leading cause of cancer death in the United States. Optical colonoscopy is the current gold standard for colorectal cancer screening and is performed over 14 million times per year in the U.S. alone. A critical task of screening colonoscopy is to identify and remove precancerous lesions, which often present as sudden elevation changes (either depressions or bumps) of the smooth surface of the colon. Lesions as small as a few millimeters in height or depth can harbor malignant potential (so called “flat lesions”). The average human colon is a tube about 1.5 meter in length and 5 cm in diameter. A major limitation in the value of screening colonoscopy is that clinically significant lesions are frequently missed due to the large search space relative to the size of the lesion, compounded by the limited time in which colonoscopies are performed to be a cost-effective screening tool. This challenge is compounded when the endoscopist is forced to rely on a two dimensional image that is obtained from a conventional colonoscope. More particularly, in conventional colonoscopy, the endoscopist must infer the morphology of these lesions from the two-dimensional images that a conventional colonoscope provides.

In conventional endoscopy, the field of view (FOV) is illuminated simultaneously from multiple sources to reduce shadowing and increase the ambient luminosity, emphasizing the color contrast for the endoscopist. However, shadows and changes in luminosity due to the varying orientation of the sample surface represent one of the visual cues that aid the human visual system in gathering information about the shape (i.e., topography) of objects. By minimizing the shadows, some of the morphologic information from the sample is irretrievably lost. To perceive the three-dimensional shape of the tissue from the two-dimensional image, the endoscopist has to rely on his familiarity with the endoscopic environment, motion perspective, and parallax. This inadequate technology is partly responsible for the fallibility of screening colonoscopy. It is estimated that 30% of clinically significant lesions are missed during routine screening. Additionally, non-polypoid lesions, particularly ones with a recessed topology, are likely to harbor malignant potential and may be missed even more frequently than polypoid lesions.

One factor limiting conventional colorectal cancer screening is that clinically significant lesions are frequently missed during a colonoscopy procedure due to subtle lesion contrast. One of the few accepted ways to increase lesion visibility is to spray a blue (or indigo) dye into the lumen to create color contrast at topographical changes in the mucosa (“chromoendoscopy”). However, although there is a consensus that it improves lesion detection rates, chromoendoscopy is too time consuming to be used in routine screening—the spraying and rinsing protocol roughly doubles the procedure time, from 15 minutes for a conventional colonoscopy, to over 30 minutes for chromoendoscopy.

Photometric stereo imaging is an established computer vision technique to calculate the surface normals of each pixel in a field-of-view from a sequence of images from a single view illuminated with different sources. Assuming a Lambertian remission of the light, the surface normal of each pixel can be calculated by solving a system of linear equations that include the measured intensity at a given pixel from each source. By integrating the associated gradients, the three-dimensional topology of the FOV can also be reconstructed. Unfortunately, conventional photometric stereo imaging operates under constraints that are impractical for endoscopy—it requires a narrow-angle FOV, and that the directional vector from each object pixel to each light source is known (a vector field which changes with every movement of the sources relative to the object). This last constraint is typically achieved by placing the light sources far away from the sample so that the directional vectors are approximately constant. Traditional photometric stereo imaging fails when the light sources are close together relative to the sample and at a short working distance with respect to the object because the relative source positions for each pixel are unknown. This limitation makes photometric stereo impractical for applications in endoscopy, especially because endoscopic systems have large field of view optic systems which exaggerate this effect increasingly away from the optical center of the images.

Despite efforts to date a need still exists for improved systems and methods for performing and utilizing three-dimensional imaging in an endoscopy system. These and other needs are addressed by the present invention.

### SUMMARY OF THE DISCLOSURE

Systems and methods are disclosed herein for performing and utilizing three-dimensional imaging in an endoscopy system. The systems and methods advantageously take into consideration geometrical factors involved in the endoscopic settings, e.g., correcting for consistent distortions introduced by the small source separation, the varying distance and direction from the sample to the sources, the varying illumination intensity in the sample, the movement of the sample between subsequent images, and/or the wide angle field of view cameras used in endoscopy.

In exemplary embodiments the systems and methods of the present invention employ photometric imaging for endoscopic applications. In particular, a photometric imaging system is disclosed including an imaging device and illumination system in a tubular endoscope body and a processor device to process image data and control system operation.

In a preferred embodiment, the method module acquiring a series of images illuminating the sample from each of a number of different light sources sequentially. This series of pictures is then used to calculate both the full illumination image, substantially equivalent to the conventional endoscopy image, and a map of the spatial orientation of the object surface for each pixel in the image. The topological information contained in the spatial orientation of the object surface can be used to compute height profiles, 3D renderings, generate conventional color images as if the object was illuminated from a fictitious source, overlay relevant morphologic information on top of the conventional image, or used as input to a computer aided detection process that finds colorectal cancer lesions based on the shape of the colon walls in addition to its color

In general, the imaging device may be configured for imaging a target surface under a plurality of different lighting conditions. Thus, in exemplary embodiments, the imaging device may include a configuration of one or more light sources for illuminating a target surface from each of a plurality of illumination directions and a detector for imaging the target surface under illumination from each of the plurality of illumination directions. In alternative embodiments, the imaging device may include a configuration of a light source for illuminating a target surface and one or more detectors for imaging the target surface from each of a plurality of detection directions. In exemplary embodiments, imaging the target surface may include high dynamic range (HDR) imaging of the target surface, e.g., by changing at least one of (i) an intensity of illumination and (ii) a sensitivity of the detector. In exemplary embodiments, implementing HDR imaging may involve merging imaging data from multiple low-dynamic-range (LDR) or standard-dynamic-range (SDR) images. In other embodiments, implementing HDR imaging may involve tone mapping to produce exaggerated local contrast. HDR imaging may be applied with respect to acquired images or with respect to information extracted from the images, e.g. to directional gradients.

Typically, the processor is operatively associated with the imaging device and configured calculate topographic information for the target surface based on the imaging of the target surface under the plurality of different lighting conditions. Thus, in exemplary embodiments, the processor may be configured to calculate a surface normal map for the target surface. While specific algorithms are provided, according to the present disclosure, for calculating a surface normal map for the target surface, it is noted that the present disclosure is not limited to such algorithms. Indeed, any conventional photometric imaging process may be used to derive topographic information from the acquired imaging information.

Importantly, the processor is typically configured to emphasize high frequency spatial components. Thus, in exemplary embodiments, the processor may be configured to emphasize high frequency spatial components, e.g., by filtering out via a high pass filter, low frequency spatial components of the derived topographic information. Thus, in exemplary embodiments, a high pass filter may be applied to a derived surface normal map of the target surface. In other embodiments, a high pass filter may be applied to directional gradients for the target surface by scaling the direction normal to the surface and high-pass filtering each of the directional gradients. In yet further embodiments, a high pass filter may be applied to individual images, e.g., each corresponding to a particular lighting condition, prior to combining the images. Alternatively, in exemplary embodiments, the processor may be configured to emphasize high frequency spatial components by detecting high frequency spatial components. As disclosed herein, the emphasis on high frequency spatial components is particular useful in an endoscopic setting, where design constraints (primarily the FOV being large relative to the distance from the target surface to the light sources) typically result in low spatial frequency error on the reconstructed normal, e.g., on the order of one cycle per FOV. Emphasis on the high frequency spatial components effectively enables accounting for the low frequency artifacts.

It is noted that emphasizing high frequency components, according to the present disclosure, is not limited to filtering out low frequency spatial components of the derived topographic information. Indeed, in alternative embodiments, emphasizing high frequency components may include applying an algorithm which identifies a high frequency surface feature, e.g., based in part on one or more parameters related to the derived topographic information.

The present disclosure also provides systems and methods for analyzing or otherwise utilizing topographical information (such as derived using the disclosed photometric imaging systems and methods or via other conventional means) in conjunction with conventional two-dimensional endoscopic imaging information, within the context of endoscopy. Thus, in exemplary embodiments a conventional two-dimensional endoscopic image may be overlaid with topographical information. In other exemplary embodiments, topographic information may be used in conjunction with conventional two-dimensional endoscopic imaging information to facilitate computer assisted detection (CAD) of features (such as lesions) on the target surface. Advantageously, the present disclosure enables detection of both topographic information and conventional two-dimensional endoscopic imaging information using a common instrument.

The foregoing and other objects, aspects, features and advantages of exemplary embodiments will be more fully understood from the following description when read together with the accompanying drawings.

### BRIEF DESCRIPTION OF THE DRAWINGS

FIG. 1 depicts and exemplary photometric imaging system according to the present disclosure, the imaging system including generally an imaging device and a processor.

FIGS. 2 and 3 depict exemplary imaging devices for performing photometric stereo endoscopy and reconstructing the normal map of the surface by comparing images of a sample taken under different illuminations.

FIGS. 4 and 5 depict exemplary prototypes of imaging devices used in testing the concepts of photometric stereo endoscopy (PSE) described herein.

FIG. 6a depicts an exemplary method for implementing photometric stereo endoscopy (PSE), according to the present disclosure.

FIG. 6b illustrates a processing sequence in accordance with preferred embodiments of the invention.

FIG. 7 depicts an exemplary applying PSE to reconstruct a surface normal map from a sequence of images of the same field of view under different illumination conditions.

FIG. 8 depicts an exemplary PSE normal map and topography estimation in a silicon colon phantom. More particularly, (a) depicts the surface normal directions and 3D rendering of a cecum view which capture the orientation of slopes and curvature, which are not contained in a conventional color image. Three diminutive bumps that are 0.5 to 1.0 mm in height are registered as elevations on the normal map (white arrows). (b) depicts the surface normal directions and 3D rendering of a tubular sample of the transverse colon. High frequency morphology which shows details of features at different working distances contained in the field of view. Cast shadow artifacts consistently exaggerate slopes from the feature generating the shadow.

FIG. 9 depicts an exemplary PSE morphology estimation for ex vivo human tissue with heterogeneous optical properties. In particular, (a) depicts reconstruction of the morphology of a polypectomy ulcer (white arrow) and surrounding tissue folds in formaline fixed colon tissue correlate with the folds that are visible in the conventional image; (b) depicts a plateau shape of a sessile polyp in the fixed ex-vivo right colon tissue, and (c) depicts a metastatic melanoma lesion in fresh ex-vivo small bowel tissue both of which are prominent in the estimated morphology.

FIG. 10A-10F demonstrating that even with a narrow light source separation system, PSE is still able to recover gradient directions of a 1 mm height, 0.5 mm radius for a 3D printed elevation at 35 mm working distance. In particular, 10A depicts conventional image captured with the modified endoscope; 10B depicts an acquired conventional color image that is ambiguous regarding the shape of a feature; 10C depicts a three-dimensional rendering based entirely on contrast and shading in the conventional color image; the surface directions; 10D depicts a photograph of the 3D printed sample; 10E provides a visual representation of numerical reference of surface directions as determined using PSE and 10F depicts the elevated morphology of the feature as determined using PSE.

FIGS. 11 and 12 depict additional exemplary configurations for imaging devices for PSE according to the present disclosure.

FIG. 13A depicts an exemplary representation of a stereoscopic image or 2.5 dimensional image visualization of the field of view, according to the present disclosure. More particularly, this side-by-side stereoscopic image can be viewed with a cross-eyed configuration, in which the left inset is displayed to the right eye, and the right inset is displayed to the left eye. This allows the visual perception of depth based on the different shading present in each inset. The field-of-view shows a view of the cecal wall in a colon phantom, where the morphology of the haustra and features can be perceived through stereoscopy.

FIG. 13B depicts an exemplary method for implementing virtual chromoendoscopy, according to the present disclosure.

FIGS. 13C-13F depict an exemplary embodiment illustrating the concept of virtual chromoendoscopy, according to the present disclosure.

FIG. 14a depicts an exemplary method for implementing CAD, according to the present disclosure.

FIG. 14b depicts an exemplary embodiment illustrating applying PSE to CAD, according to the present disclosure.

FIG. 15 depicts an exemplary computing device, according to the present disclosure

FIG. 16 depicts an exemplary network architecture, according to the present disclosure.

FIG. 17 illustrates a process sequence for processing image data in accordance with the disclosure.

FIGS. 18A and 18B illustrate preferred embodiments of an endoscope system in accordance with the disclosure.

FIGS. 19A and 19B illustrate illumination fields in accordance with preferred embodiments of the disclosure.

FIGS. 20A and 20B illustrate endoscope systems in accordance with preferred embodiments of the disclosure.

FIG. 21 depicts the surfaces reconstructed by PSE before and after removing specular reflections, in accordance with preferred embodiments of the disclosure.

FIGS. 22 and 23 compare images obtained from VCAT and conventional chromoendoscopy, in accordance with preferred embodiments of the disclosure.

FIGS. 24A and 24B depict exemplary self-contained imaging devices for implementing PSE, in accordance with preferred embodiments of the disclosure.

FIG. 25 depicts topographic information acquired including surface texture and vasculature features, in accordance with preferred embodiments of the disclosure.

### DETAILED DESCRIPTION

The present invention relates endoscopic imaging techniques referred to herein as photometric stereo endoscopy (PSE). According to the present invention, PSE generally involves systems and methods which enable acquisition of high-spatial-frequency components of surface topography and conventional two-dimensional images (e.g., color images). Thus, in exemplary embodiments, the orientation of the surface of each pixel in the field of view can be calculated using PSE. This orientation can be represented, e.g., by a surface normal, surface parallel vector, or an equation of a plane. In some embodiments, a resulting surface normal map can optionally be reconstructed into a surface topography. Advantageously, PSE allows for implementation with an imaging device conforming to an endoscopic form factor.

In exemplary embodiments, PSE enables accurate reconstruction of the topographical information relating to small features with complex geometries and heterogeneous optical properties. Thus, in some embodiments, PSE enables accurate reconstruction of the surface normal for each pixel in the field of view of an imaging system. By emphasizing high-frequency spatial components PSE can capture spatial information of small features in complex geometries and in samples with heterogeneous optical properties. This normal map can then be reconstructed into a surface topography. Results obtained with ex vivo human gastrointestinal tissue demonstrate that the surface topography from dysplastic lesions and surrounding normal tissue can be reconstructed. Advantageously, PSE can be implemented with modifications to existing endoscopes, and can significantly improve on clinically important features in endoscopy. Thus, in exemplary embodiments, PSE can be implemented using an imaging device characterized by a single detector and multiple illumination sources. Moreover, the image acquisition and processing techniques described herein are fast thereby facilitating application in real-time.

One of the purposes of the systems and methods disclosed herein is to enable three-dimensional surface imaging through a small diameter endoscope to decrease the frequency of missed lesions in endoscopy screening. Photometric Stereo Endoscopy (PSE), allows for conventional two-dimensional image information and topographical information to be obtained simultaneously, using a single device. This technology provides important information to an endoscopist such as the topology, and especially the high-frequency topology of the field of view. Thus, PSE equips the endoscopist with valuable, previously unavailable morphology information. Two other key features of PSE are: (1) it can be implemented without altering the conventional images that the endoscopist is used to, and (2) it can be implemented using an all optical technique with automated image processing. Topographical information obtained using PSE can also be used to enable improved computer aided diagnosis/detection (CAD) and virtual chromoendoscopy.

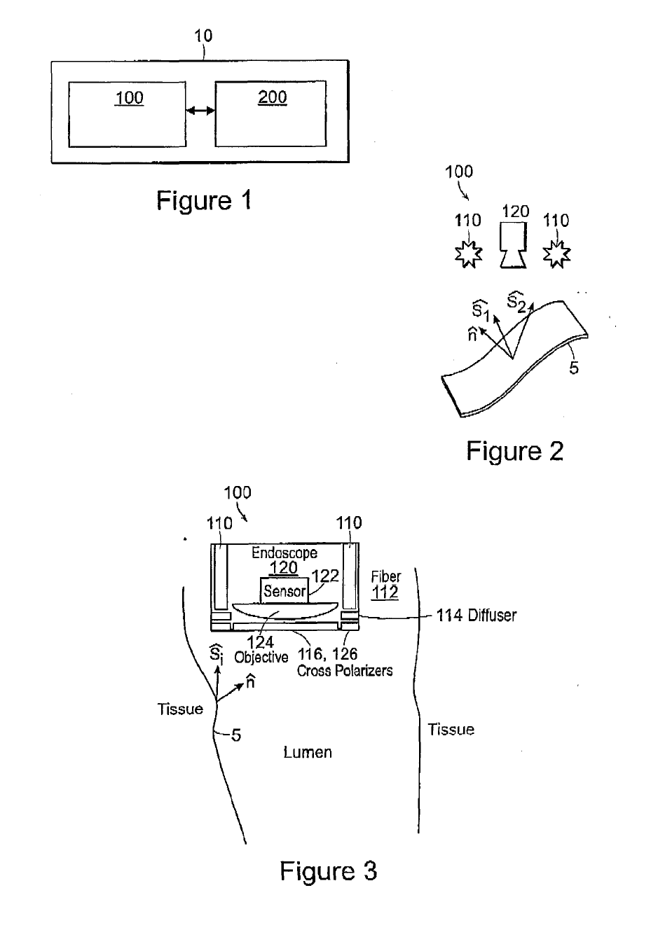

With reference to FIG. 1, an exemplary photometric imaging system 100 is depicted. The exemplary imaging system 10 includes an imaging device 100 configured for imaging a target surface under a plurality of different lighting conditions and a processor 200 configured for processing imaging information from the imaging device for the plurality of different lighting conditions to calculate topographic information for the target surface, wherein the calculated topographic information emphasizes high frequency spectral components, while deemphasizing low frequency spectral components. As described herein, imaging system 10, may be used to implement PSE.

In exemplary embodiments, a cut-off frequency of 0.1 cm⁻¹ may be used to isolate high frequency components (e.g., for imaging and analysis of lesions). In other embodiments, a cut-off of 1 cm⁻¹ may be used to isolate high frequency components (e.g., for imaging and analysis of crypts and pits). In yet other embodiments a cut-off frequency of 8 cycles per field of view may be utilized.

In exemplary embodiments, PSE may involve calculating the surface normal of each pixel in an image from a set of images of the same FOV taken with different lighting. FIGS. 2 and 3, depict exemplary imaging devices 100 for obtaining images of a target surface 5. In such embodiments, the direction normal to the surface may be represented by \hat{n}, the direction to light source i may be represented by the vector [symbol] , and the image intensity may be proportional to cos θ=\hat{n}· [symbol] . under different illumination conditions. Each exemplary imaging device 100 includes a plurality of light sources 110 and a detector 120. With specific reference to FIG. 3, it is noted that the imaging device 100 may be adapted to conform to an endoscopic form factor. FIG. 3 also illustrates exemplary components for a light source 110 including fiber optics 112, a diffuser element 114 and a cross polarizer 116 and exemplary components for a detector 120 including a sensor 122, optics 124 and a cross polarizer 126. The use of a diffuser element and cross polarizers advantageously provides diffuse illumination across a wide FOV, reduces specular reflections and enhances contrast and saturation (e.g., by reducing saturation) in the resulting images.

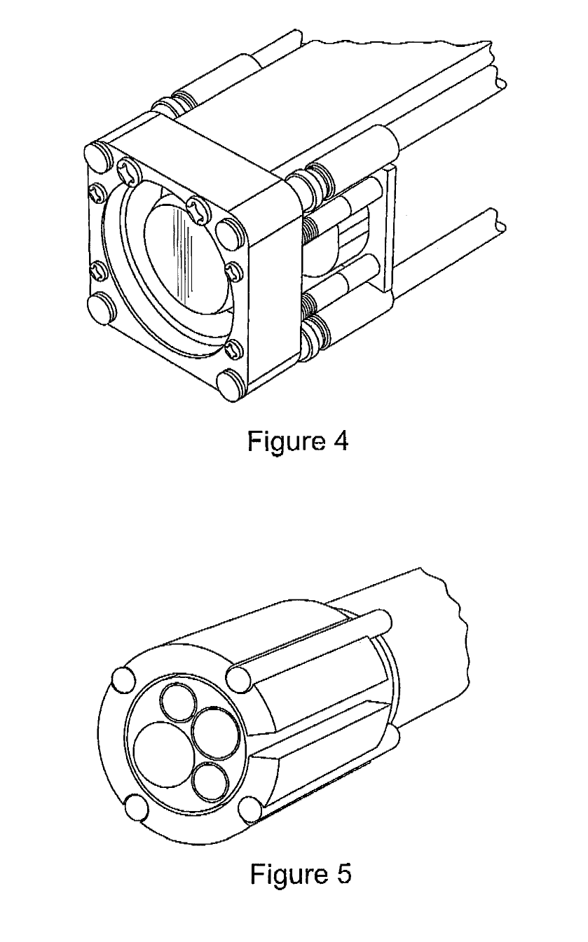

While the illustrated embodiments of imaging device 100 depicted in FIGS. 3 and 4 include a plurality of light sources and a single detector the present disclosure is not limited to such embodiments. Indeed in other embodiments, the imaging device may include a single light source and a plurality of detectors. In yet further exemplary embodiments, the imaging device may include a single detector and a single light source wherein the detector or light source may be moved relative to the other to generate different illumination conditions. Notably, exemplary embodiments can be advantageous to maintain a common FOV to allow for easy indexing of images. Thus, single detector embodiments, e.g., with either a plurality of light sources or a single moving light source, may be particularly advantageous.

FIGS. 4 and 5, depict two exemplary imaging devices which were used to evaluate the systems and methods disclosed herein. FIG. 4 illustrates a preferred embodiment while FIG. 5 illustrates a modified commercial endoscope. The system of FIG. 5 was used to measure illumination and image capture controls. In particular, this system, was used because of its ability to access raw image data from a sensor, synchronize source illumination with the frame rate, and introduce cross-polarizers to reduce specular reflections. However, the source separation was 35 mm, which can be reduced to a system having an endoscope body with a diameter of 5-20 mm. The distal tip of typical commercial colonoscopes ranges in diameter from 11 to 14 mm (for example 13.9 mm in the CF-H180AL/I model, Olympus). Note that in exemplary embodiments, PSE may be implemented using a conventional commercial colonoscope (such as CF-H180AL/I model, Olympus), e.g., modified by attaching external light sources with a sheath.

PSE was also implemented using a gastroscope modified by attaching external light sources with a sheath. See FIG. 5. Using the modified gastroscope in this embodiment, the source separation was reduced to below 14 mm. In this embodiment, the gastroscope had an initial 10 mm diameter, which was modified by attaching light sources via a sheath which added 4 mm to the diameter resulting in a 14 mm diameter However, there were several limitations with the commercial system. First, because the interface between the Pentax sensor and digitization hardware was inaccessible, only images that have been post-processed by the commercial system were accessed, and because of the small size of the endoscope, cross-polarizers to reduce specular reflections were not incorporated into this embodiment. The PSE system demonstrated an ability to accurately acquire the topography from small features (1 mm in height or depth) at typical working distances used in endoscopy (10-40 mm).

The system was constructed with four light sources mounted around a camera with a fish-eye lens. The size of the housing was 30 mm×30 mm, and the four sources were oriented at equal angles about a circle with a 35 mm diameter. A Dragonfly®2 remote head camera was used with a ⅓″ color, 12-bit, 1032×776 pixel CCD (Point Grey Research, Inc.). The images were created with a 145° field-of-view board lens (PT-02120, M12 Lenses). White LEDs were used for illumination (Mightex FCS-0000-000), coupled to 1 mm diameter, 0.48 NA multimode fibers. Sources were synchronized to the camera frame rate of 15 Hz. A holographic light shaping diffuser was placed at the end of each source to efficiently spread illumination light (Luminit). Linear polarizers were placed in front of the sources and objective lens in a cross-configuration to minimize specular reflection. Images in raw data format were processed with a de-mosaicking interpolation process to provide full resolution RGB images from Bayer-patterned raw images. The pixel intensities were then estimated by a weighted average of the three color channels.

Turning to the modified commercial endoscope system, a Pentax EG-2990K gastroscope with a Pentax EPK-1000 video processor was used. For illumination, fibers with an integrated light diffuser (Doric Lenses Inc.), and no polarization filters were used. The 4 fibers were secured at equal angles in a 12 mm diameter circle around the endoscope tip, making an external diameter of 14 mm. Components can be held within a flexible plastic tube or sheath. Uncompressed video in NTSC format was aquierd at 8 bit, 720×486 pixel resolution, 29.97 interlaced frames per second using a video capturing device (Blackmagic Intensity Shuttle). Light sources were alternated at 60 Hz, synchronized with the video signal to deinterlace a sequence of RGB frames captured with only one light source active at a time. Frames were then interpolated in every other horizontal line to obtain full resolution images. The image intensity was estimated as the weighted average of the three color channels. Note that in certain embodiments, the camera was not positioned at the center of the circle that the four sources are co-located about. Rather the camera was off center, and the source vector that was used for each pixel's normal calculation, took that into account. In exemplary embodiments PSE may be implemented using a sensor which is equidistant from each of the light source(s). In other embodiments, the sensor and/or light source(s) may be unevenly spaced relative to one another.

In implementing PSE using the endoscope devices of FIGS. 4 and 5, an exemplary process was applied for processing imaging data. The applied process can use the approximation that the light remitted from the sample surface follows the Lambertian reflectance model. In exemplary embodiments, other more sophisticated models can be used, including, e.g., a Phong model, or ones that take into account both shadowing and specular reflections. See, e.g., Svetlana Barsky, Maria Petrou, “The 4-Source Photometric Stereo Technique for Three-Dimensional Surfaces in the Presence of Highlights and Shadows,” IEEE Transactions on Pattern Analysis and Machine Intelligence, vol. 25, no. 10, pp. 1239-1252, October 2003, Adam P. Harrison, Dileepan Joseph, “Maximum Likelihood Estimation of Depth Maps Using Photometric Stereo,” IEEE Transactions on Pattern Analysis and Machine Intelligence, vol. 34, no. 7, pp. 1368-1380, July 2012, Satya P. Mallick, Todd Zickler, David J. Kriegman, and Peter N. Belhumeur, “Beyond Lambert: Reconstructing Specular Surfaces Using Color.” Proc. IEEE Conf. Computer Vision and Pattern Recognition, June 2005, K. Ikeuchi, “Determining a depth map using a dual photometric stereo system,” Int. J. Robotics Res. 6, 15-31 (1987), Tai-Pang Wu, Chi-Keung Tang, “Photometric Stereo via Expectation Maximization,” IEEE Transactions on Pattern Analysis and Machine Intelligence, vol. 32, no. 3, pp. 546-560, March 2010, Alldrin, N., Zickler, T., & Kriegman, D. (2008, June). Photometric stereo with non-parametric and spatially-varying reflectance. In Computer Vision and Pattern Recognition, 2008. CVPR 2008. IEEE Conference on (pp. 1-8). IEEE, Georghiades, A. S. (2003, October). Incorporating the Torrance and Sparrow model of reflectance in uncalibrated photometric stereo. In Computer Vision, 2003. Proceedings. Ninth IEEE International Conference on (pp. 816-823). IEEE, and Chung, H. S., & Jia, J. (2008, June). Efficient photometric stereo on glossy surfaces with wide specular lobes. In Computer Vision and Pattern Recognition, 2008. CVPR 2008. IEEE Conference on (pp. 1-8). IEEE, each of which are incorporated herein by reference. Several variations can be introduced by using different reflectance models. In general, a different number of measurements allows calculating the normal vectors and/or albedo more precisely or under different model assumptions.

The Lambertian reflectance model describes materials with a diffusely reflecting surface and isotropic luminance. This means that their apparent brightness or luminous intensity l is proportional to the surface irradiance l₀, the reflection coefficient or albedo A and to the cosine of the angle between the unit vector normal to the surface \hat{n} and the unit vector indicating the direction to the illumination source ŝ. This relation is represented as:

l∝Al₀ŝ·\hat{n}. (1)

Neglecting cast shadows and specular reflections, a constant, α is defined, such that it includes the proportionality factor, the irradiance, and the albedo of the surface are imaged in a given pixel. When the light source i is on, the source direction is represented as ŝᵢ and the measured intensity mᵢ at that pixel can then be represented as:

mᵢ=ŝᵢ·\vec{n}, (2)

where \vec{n}=[nₓ,n_{y},n_{z}]^{T} is a non-unitary vector with magnitude α and direction \hat{n}. An example sequence of images under different illumination is shown in FIG. 2 a-d.

A sequence of three measurements of the same sample can be written as:

$$[ \begin{matrix}{s}_{x 1} & {s}_{y 1} & {s}_{z 1} \\ {s}_{x 2} & {s}_{y 2} & {s}_{z 2} \\ {s}_{x 3} & {s}_{y 3} & {s}_{z 3}\end{matrix} ] [ \begin{matrix}{n}_{x} \\ {n}_{y} \\ {n}_{z}\end{matrix} ] = [ \begin{matrix}{m}_{1} \\ {m}_{2} \\ {m}_{3}\end{matrix} ] . \qquad ( 3 )$$

This is a linear system of equations that can be solved for \vec{n} if the light sources matrix is non-singular. This condition is equivalent to requiring that the three light sources and the sample do not lie in the same plane. If more than three measurements are acquired, the normal vectors can be estimated by minimizing the residual error given the measurements and the source directions:

$$arg {min}_{{n}_{x} , {n}_{y} , {n}_{z}} \sqrt{{\sum }_{i} {( {\hat{s}}_{i} \cdot \vec{n} - {m}_{i} )}^{2}} . \qquad ( 4 )$$

The traditional photometric stereo assumption that ŝᵢ is constant for all pixels in the image becomes especially inaccurate when the FOV is large relative to the distance from the object to the light sources. It was determined that the variable nature of ŝᵢ induced low frequency error on the reconstructed normals, on the order of 1 cycle per FOV. In particular, low frequency artifacts resulted from the slow changing of the source directions across the field of view. As noted above, one of the primary motivation for PSE is to obtain useful information about the lesions and texture present in an endoscopic setting, which are often high frequency topographies. Thus, the derived topographic information is processed, e.g., by applying a high pass filter, to emphasize high frequency components over the inaccurate low frequency artifacts.

Assuming a continuous sample that can be described as z=f(u,v) with z the distance from the objective to the sample and (u,v) the pixel coordinates, its directional gradients can be obtained by scaling the direction normal to the surface:

$${[ \begin{matrix}- \frac{\partial f}{\partial u} & - \frac{\partial f}{\partial v} & 1\end{matrix} ]}^{T} = \frac{\vec{n}}{{n}_{z}} = {[ \begin{matrix}\frac{{n}_{x}}{{n}_{z}} & \frac{{n}_{y}}{{n}_{z}} & 1\end{matrix} ]}^{T} \qquad ( 5 )$$

Because both the spatial frequency filter and the differentiation are linear operations on (u,v), these operations are interchangeable, and the high-pass filter of −∂f/∂u is equivalent to the gradient in direction u of the high-passed surface. Thus, by high-pass filtering each of the directional gradients, one can obtain the gradients of the high frequencies of the shape. For each directional gradient, a high pass filter may be applied by subtracting the low frequency component of the signal, which is calculated as a convolution of the original gradient with a Gaussian kernel with σ=40 pixels in image space. As applied with respect to the present embodiments, this filter's full width at half maximum value was approximately 8 cycles per FOV.

To calculate height maps, the filtered gradients can be integrated using a multigrid solver for the Poisson equation that minimizes integration inconsistency errors. See T. Simchony, R. Chellappa, and M. Shao, “Direct analytical methods for solving Poisson equations in computer vision problems,” IEEE Transactions on Pattern Analysis and Machine Intelligence 12(5), 435-446 (1990); and D. Scaramuzza and R. Siegwart, “A Practical Toolbox for Calibrating Omnidirectional Cameras,” in Vision Systems: Applications, ISBN 978-3-902613-01-1 Chapter 17, Swiss Federal Institute of Technology (2009), the entire contents of these references being incorporated herein by reference. To visualize both the color information and the acquired topography, one can overlay the color image on the calculated height map.

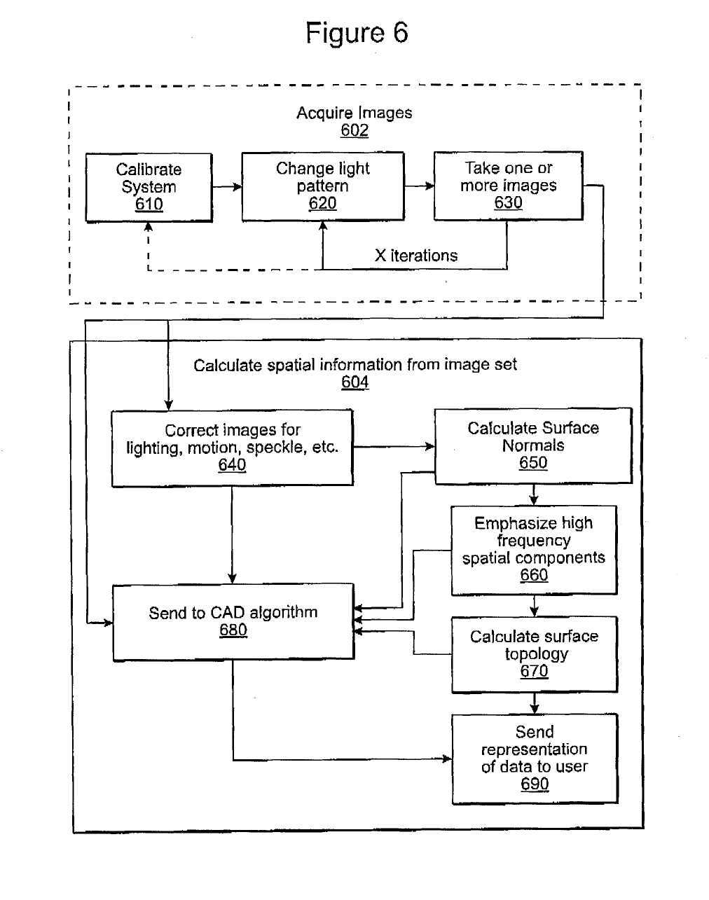

With referent to FIG. 6A an exemplary method 600 for implementing photometric stereo endoscopy (PSE) is depicted. The exemplary method generally includes steps of acquiring imaging information 602 and calculating spatial information from the acquired images 604. According to exemplary embodiments step 602 may generally include acquiring a series of images, e.g., for a common FOV, each under different illumination conditions, e.g., by achieved by illuminating the sample from sequentially using different light sources Similarly, step 604 may generally include using the series of images to calculate topographic information for the sample, e.g., a surface normal map representing the spatial orientation of the object surface for each pixel. In a more detailed fashion, a typical embodiment of method 600 may include a subset of one or more of the following steps: Calibrating the system 610, sequentially changing the illumination conditions 620 and acquiring one or more images 630 for each illumination condition, preferably for a common FOV, pre-processing the acquired images to correct for lighting, motion, speckle, etc. 640, calculating surface normals for the surface 650, emphasizing high frequency spatial components 660, calculating surface topography 670, and utilizing the calculated topography information, e.g., in CAD application 680 or create visualizations for the doctor 690.

In yet further embodiments, process 600 may involve implementing the following steps illustrated in connection with the embodiment illustrated in the method 750 of FIGS. 6B(i) and 6B(ii): First, the imaging system is calibrated 752 such that its parameters related to translation of coordinates between image space and object space, are known. Next, calibrate 754 the illumination system measuring the intensity irradiating from each light source as a function of the object space. System of multiple illumination sources is actuated 756 with a controller where more than one electromagnetic radiation sources capable of irradiating the object from different originating positions and/or different wavelengths, or a combination of positions and/or wavelengths and a switching and/or synchronization method that allows using a different illumination source for each image in a sequence. Then acquire 758 a series of images illuminating the sample from each different light source sequentially and register 760 images in the sequence of images acquired when a relative movement between the camera and the sample takes place between subsequent acquisitions. Extract 762 the low spatial frequency component from each image, divide 764 each image by its low frequency component and use 766 the result of this division for each image to find a common transformation mapping between coordinates of a subset of images in the acquired sequence by
i. Using an entropy based method, such as mutual information, and
ii. Using an intensity difference based method, such as mean square error. Use the series of images to calculate 768 a map of morphological information of the object surface for each pixel by computing 770 a different projection of the image using the camera distortion parameters, processing 772 the images to reduce specular reflection artifacts and computing 774 intensity map of the image by averaging of color channels, adjusting luminance channel from luminance and chromaticity color space and using raw data with Bayer filter pattern. Next, extract 776 the high spatial frequency component of the image by calculating illumination vectors.

The calculation 778 is performed by first assuming that the process is identical for all pixels in which a point spatial position of the sample is used to calculate the spatial direction from this point to each source. Calculate light direction for each pixel where a spatial position of the sample for each pixel is used to calculate the direction from each point to each source. The sample surface orientation is then computed 780 and represented by a three component vector normal to the sample surface for each pixel in the image where a linear system of equations is solved relating the measured intensities, the source directions and the normal vector for each pixel, errors are minimized in the estimation of the normal vector for each pixel given the source directions and measured intensities. Next, correct the normal vectors 782 accounting for distortions caused by the varying light source directions in short working distance between the imaging system and the sample where camera calibration parameters can be used as well as illumination calibration parameters. Correct the normal vectors accounting 784 for distortions caused by different illumination magnitudes between each of the different light sources in the sequence for each pixel in the image, again using camera calibration parameters and illumination calibration parameters.

Extract 786 selective spatial frequency information from the resulting morphology maps by computing a spatial high pass filter of the morphology and computing a selective spatial frequency filter of the morphology, adapted to a specific lesion type or size. The object's surface shape can then be computed 788 and the computed surface morphology is used to recalculate light source directions for each pixel and iteratively repeat steps from intensity computation mapping step (774) onwards. Three dimensional representation of the morphology is displayed and stored 792 and an enhanced conventional image can also be displayed and stored 794 in memory.

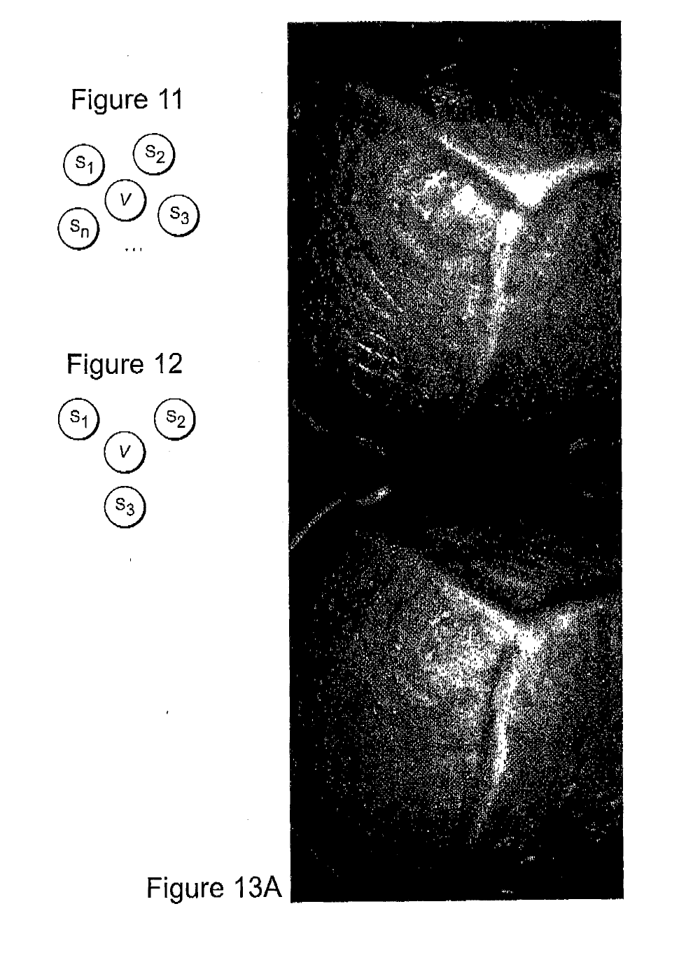

In exemplary embodiments, an imaging device suited for PSE may include more than one independently operated electromagnetic radiation source. The diagram in FIG. 11 shows a system with one camera viewpoint labeled V and a number of illumination sources enumerated {s₁, s₂, s₃, . . . sₙ}. A system with two sources is able to “see” a one dimensional orientation measure of the surface in the direction determined between the two sources. In exemplary embodiments, the projection of the surface normal vector about the plane containing the two sources and the object pixel can be determined. This information is sufficient to generate a stereoscopic image of the field of view, that is, one with not all the three dimensional information, but a 2.5 dimensional image that enables visual perception of the three dimensional information of the object. Images illuminated from three sources provide sufficient information to compute the normal orientation of the object's surface in ideal conditions with a simple reflectance model. More than three illumination sources provide additional information that can be used to resolve more unknowns in a more complex reflectance model (e.g., specular reflection-based models and bi directional reflection function-based models), or to make the simple calculations more robust to measurement noise.

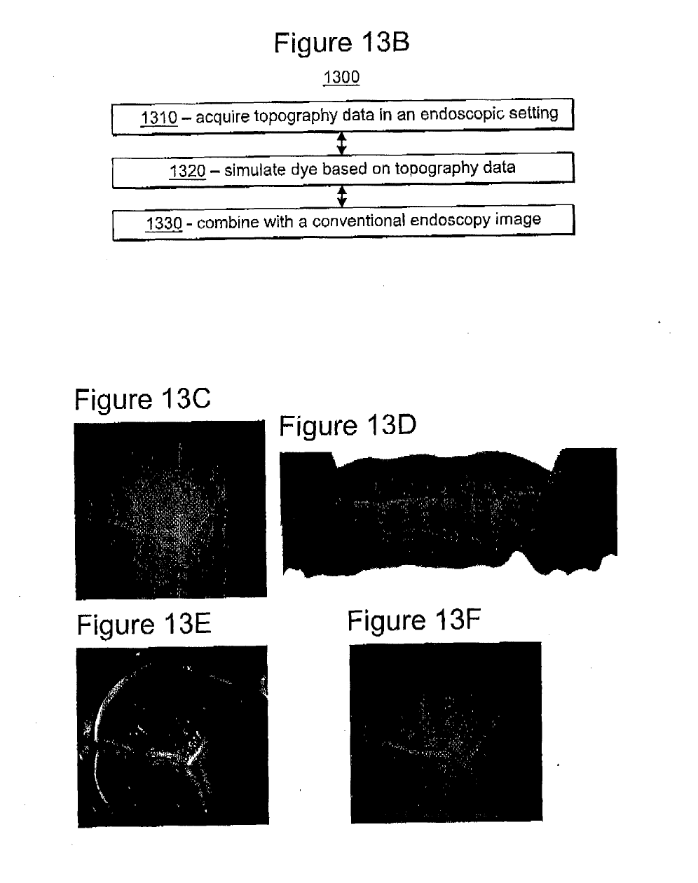

In a further exemplary embodiment, a stereoscopic image of the field of view or 2.5 dimensional image visualization can be generated with a simplified computation. If the separation of the two light sources is adequate, the luminance channels of two differently illuminated images (from the left and from the right of the field-of-view relative to the viewer) can be high pass filtered to retain the high spatial frequencies present in those luminance channels. These filtered luminance channels can be combined with the saturation and hue channels of an average color image of both differently illuminated measured images, to produce left- and right-shaded images respectively. In this way, the color is preserved from the average image and the luminance has the shading corresponding to the high spatial frequency morphology features present in the referred left and right illumination images. The resulting combinations can be presented to the left and right eye separately, to stimulate the visual perception of depth by enhancing the visual cue known as “shadow stereopsis”, See Medina Puerta, A. (1989). The power of shadows: shadow stereopsis. JOSA A, 6(2), 309-311, the entire contents of this reference being incorporated herein by reference. If the separation of the light sources is not adequate, the orientation of the surface can be computed, and pleasant left- and right-illumination shadings can be rendered from a PSE high frequency morphology estimation. As depicted in FIG. 13A, this allows the visual perception of depth based on the different shading present in each inset.

In a further exemplary embodiment, a Scanning Fiber Endoscope (SFE) can be used to obtain raw photometric stereo images. Recent advances in miniaturization of endoscopic imaging systems have allowed to perform color video imaging through a single optic fiber. The SFE system is substantially thinner than flexible endoscopes used in colonoscopy, allowing for ultrathin clinical applications. Furthermore, using a convenient arrangement of one or more illumination sources and one or more detectors, multiple images with differing apparent lighting directions can be collected. As detailed in reference [U.S. Pat. No. 6,563,105], these images allow calculating a photometric stereo estimation of the surface normal directions. As images obtained using a SFE system are affected by all the endoscopic geometry distortions described in this invention, they are also affected by the same low spatial frequency distortions. Therefore, these images can be conveniently used for the purpose of this invention, by using a high spatial frequency filter that would remove their low spatial frequency artifacts. By these means, SFE images that provide distorted photometric stereo approximations can be used with the PSE approach described in this invention, to generate representative high frequency topographic maps of the tissue surface.

Multiple variations of the way in which the illumination is switched can be considered. In a simple example, depicted in the diagram of FIG. 12, an imaging system may include a camera in the center depicted by V and there are three white light sources in different positions depicted by s₁, s₂ and s₃. A series of images can be acquired turning one light on at the time of acquisition of each image in the sequence, as indicated in Table 1.

TABLE 1

| | S₁ | S₂ | S₃ |
|---|---|---|---|
| Image 1 | On | | |
| Image 2 | | On | |
| Image 3 | | | On |

In another example, two lights may be turned on for each acquired image, as shown in Table 2.

TABLE 2

| | S₁ | S₂ | S₃ |
|---|---|---|---|
| Image 1 | On | On | |
| Image 2 | | On | On |
| Image 3 | On | | On |

In a further example, the three light sources can be turned on with different wavelengths, namely red, green and blue, and these lights are turned on with a different wavelength for each image in the sequence as summarized in Table 3.

TABLE 3

| | S₁ | S₂ | S₃ |
|---|---|---|---|
| Image 1 | Red | Green | Blue |
| Image 2 | Blue | Red | Green |
| Image 3 | Green | Blue | Red |

In a further example, the three lights of the PSE system could be turned on once with white light and once with color coded light in a sequence shown in Table 8:

TABLE 8

| | S₁ | S₂ | S₃ |
|---|---|---|---|
| Image 1 | White | White | White |
| Image 2 | Blue | Red | Green |
| Image 3 | White | White | White |
| Image 4 | Blue | Red | Green |

Images taken with white light may be used to estimate the luminance and color of the object. Images where each light has a different color may be used to retrieve the normal topographical information, since each color channels contains the information obtained from a different illumination source. The color of the object can be used to normalize the intensities obtained for the normal map with the color illuminations.

Other variations can be used, for example when the acquired series of images corresponds to a sequence of even and odd interlaced video frames, illumination sources may be turned on during the acquisition of each full frame, or they may be synchronized to switch illuminations for each half-frame of the interlaced video. In yet other embodiments, multiplexing can be used to decouple simultaneously detected signals for individual light sources, e.g., by encoding and detecting unique signatures.

Specular reflections produce portions of the acquired image to be saturated due to a high proportion of light reflected by the sample in the same direction. Image saturation is a non-linear effect that can lead to erroneous results when using the standard general assumption that the measured intensity in each pixel is proportional to the intensity of light diffused from the sample in a position corresponding to the pixel. One method of reducing the specular reflections is to have the electromagnetic emission of the sources and the detection of the imaging system in orthogonal polarization modes, so that light that is specularly reflected will not be detected due to the symmetrical preservation of its polarization upon reflection and its cancelation before detection. Light that is diffusively reflected in the surface of the sample will lose and randomize its polarization, enabling it to be detected. A different method can rely on the dampening of optical interfaces to avoid specular reflection, for example, by filling the transmission medium with water instead of air, effectively reducing the specular reflection by eliminating the air/tissue interface.

When a color CCD camera is used that utilizes a Bayer mask to filter colors into different detector units, raw images may be pre-processed using a demoisaicking algorithm that interpolates the colors in the missing pixels and computes a full resolution RGB image from the raw image. This allows calculating the conventional endoscopy color image. Photometric stereo imaging can then be computed using the luminance of the color picture or the mean intensity of the three color channels. Alternatively, raw images with the Bayer pattern may be used to compute photometric stereo for each pixel with the information of its respective color, remaining the demosaicking step only to calculate a conventional color image as is commonly performed.

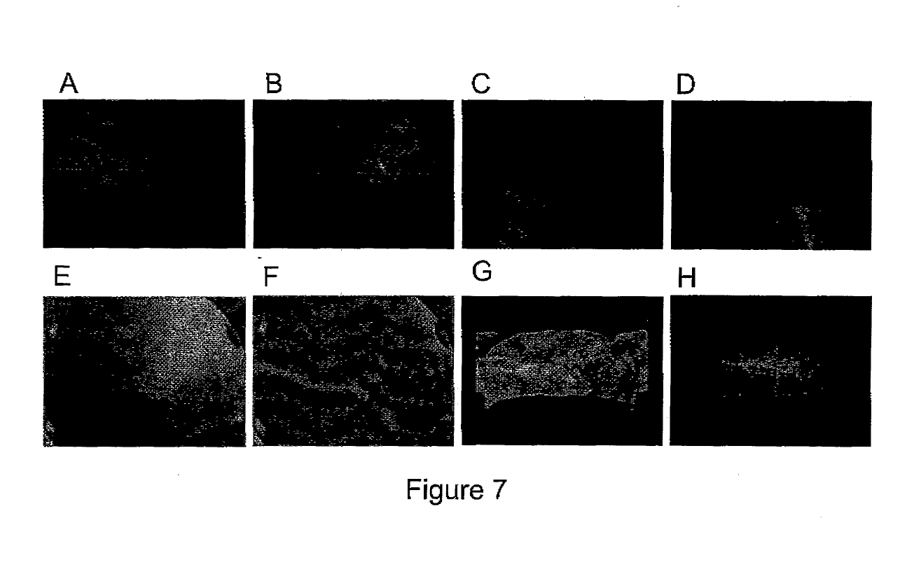

With reference to FIGS. 7A-7H, an exemplary application of PSE to reconstruct a surface normal map from a sequence of images of the same field of view under different illumination conditions (FIGS. 7A-7D), is depicted. Conventional photometric algorithms result in low frequency artifacts due to errors in the source direction vectors (FIG. 7E). Filtering out those low frequency artifacts, e.g., PSE can acquire high-frequency spatial features with potential clinical relevance (FIG. 7F). Using these normal maps, one can reconstruct the topography of the field of view (FIG. 7G) and overlay the conventional image to simultaneously present color and spatial information (FIG. 7H). Topography can be viewed at arbitrary angles and lighting conditions to improve contrast for the endoscopist.

As depicted in FIG. 3, one important aspect of endoscopy is the ability to image in a tubular environment. Thus, a silicon colon phantom was used to measure PSE imaging in a tubular environment (Colonoscopy Trainer, The Chamberlain Group). This phantom had previously been used in a study for investigating lesion detection rates in colonoscopy. The overall shape of the colon including curvature and haustra were represented in the phantom. Fabrication details provided features comparable in size to subtle colon lesions. The material had a homogeneous color, and the surface was smooth and shiny. This model served the purpose of emulating the geometry of the colonoscopy environment to evaluate effects such as the tubular shape, wide FOV, cast shadows, varying working distance and non-uniform illumination. A second phantom with a variety of bump heights and depressions was also created using a stereolithography three-dimensional printing service (Quickparts.com). This phantom enabled assessment of PSE sensitivity to height changes as a function of working distance. The phantom was painted with pink tempera paint to reduce specular reflection.

Ex-vivo Human Tissue samples were also used in conducting imaging procedures. Specimens from colonic resections (for any indication) were identified and, specimens with abnormalities were selected for imaging. All tissue samples were imaged within 24 hours of resection, either fresh or after preservation in formalin for less than 24 hours.

Both phantoms and the Ex-vivo Human Tissue samples were used in evaluating the effectiveness of PSE. The measurements thereof are described in greater detail in the sections which follow:

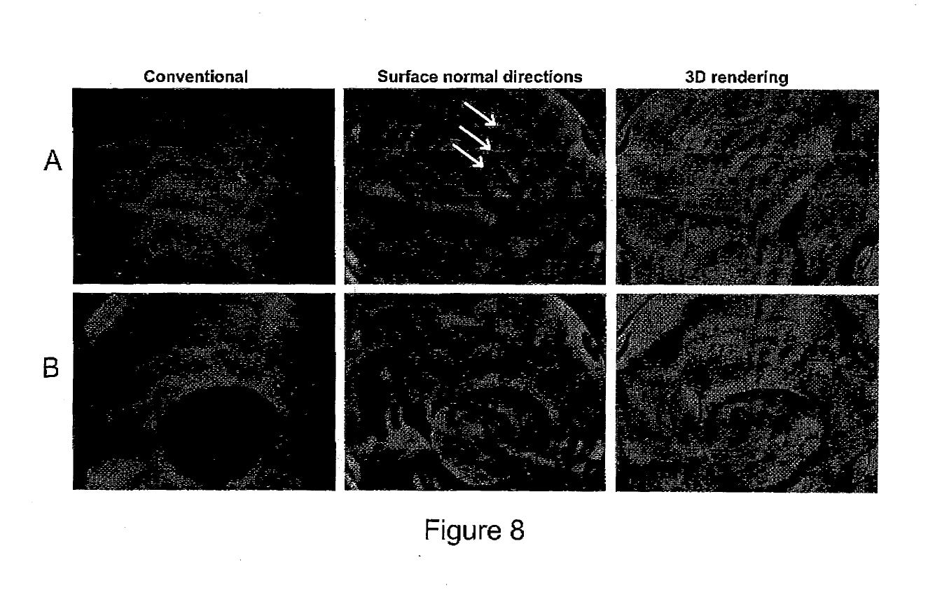

PSE imaging was performed on several regions of the silicon anatomical phantom using the bench top prototype of FIG. 4. The expected orientations were recovered for the surface across the FOV, as shown in the frontal view of the cecal wall in the silicone phantom presented in FIG. 8 (a). As depicted in FIG. 8, the reconstructed surface normal map may be visualized using a standard computer vision technique, where the surface normal is normalized and x, y, and z components of the vector are mapped to values of red, green, and blue, respectively. The flat regions of the cecum generate regions with normal components pointing primarily in the z-direction, and bumps and ridges create normals that are correctly reconstructed after integration. It is important to note that the topographical data presented in the surface normal map and the 3D rendering are complementary to the color information in the conventional image as this topography cannot be reconstructed from the conventional image alone. Three diminutive bumps that are each 0.5 to 1 mm in height are registered as elevations in our reconstruction, though it is difficult to appreciate based on the conventional color image alone (see FIG. 8a).

As previously discussed, the illumination intensity reaching the sample from the light sources is strongly affected by the working distance, which can vary significantly within the FOV. For example, when imaging down a tubular shape, pixels in the center of the image receive much less light than those at the periphery. However, accurate normal reconstruction in PSE relies on intensity differences for each pixel in a series of images, and lighting changes that are consistent across the PSE image series should only affect the signal intensity over noise. This concept is demonstrated in a PSE image of the transverse colon in FIG. 8 (b). Though the light intensity reaching the surface down the tube is much lower than that illuminating the adjacent wall, the high-frequency surface orientations of the object are still acquired.

There are several sources for error that are pronounced in a tubular geometry. The assumption that the source vectors are constant across the FOV can become worse as the distance between each point in the object and the light source changes. Furthermore, any portion the object that is shadowed differently by different light sources creates a nonlinear artifact: the region that is cast in shadow is reconstructed to have a surface normal that points more perpendicularly to the direction of the light source that shadows the region than it should. This artifact exaggerates slopes facing away from the lights. Qualitatively, this effect emphasizes ridges and sharp features, which may actually be helpful for the purpose of increasing lesion contrast. In FIG. 8 (b) this effect is observed in the shadows cast by the muscular features and haustra of the simulated colon.

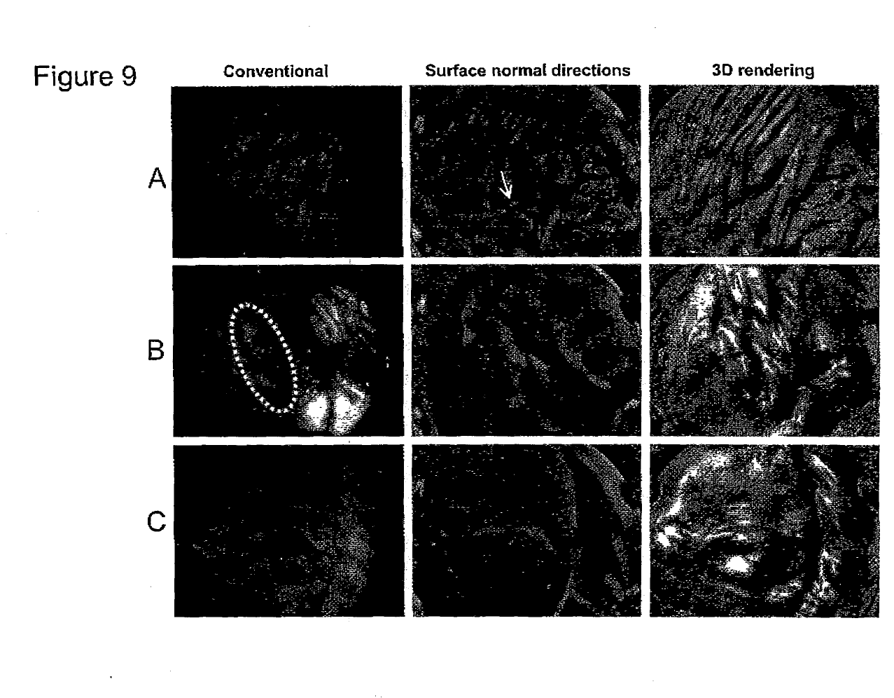

The system was used to perform PSE on ex-vivo human gastrointestinal tissue in order to evaluate performance on samples with heterogenous optical properties, reflective surfaces, and clinically-relevant lesions. FIG. 9 (a) presents the topography obtained from a right colectomy with a tattoo applied next to an ulcer that resulted from a polypectomy. Here, our normal map correlates to the visible folds in the conventional image. The ulcer, identified by a gastroenterologist at the time of the imaging, was reconstructed as a prominent indentation in the tissue. However the tattoo, which left a concentrated point of indigo color at the site of the injection, did not register as a topographical change. This illustrates that PSE is able to separate a pixel's surface normal vector from its albedo.

Next, a sessile lesion that was identified after a right colectomy was imaged (FIG. 9 b). In this measurement, the light source in the bottom right of the FOV did not diffuse as well as the other three sources. As a result, the image with this light source on saturated the bottom right of the FOV and the topologies were poorly reconstructed in that region. Nonetheless, the sessile lesion here clearly influences the normal map. Looking at the surface rendering that was generated from the normal map, the lesion did have the same plateau-like topography that is characteristic of a sessile lesion, and that was observed during this measurement.

Finally, a metastatic melanoma that was present in fresh ex-vivo human small bowel tissue was imaged (FIG. 9 c). This feature is also identifiable in the normal map and reconstructed height profile. Note again that here PSE is able to distinguish between color changes of the tissue and actual folds that are present in the tissue.

Because the ex-vivo human tissue was wet, specular reflection was more prominent than was observed in the silicon phantom. This led to artifacts in our surface normal reconstructions. Specifically, pixels that have specular reflections are reconstructed to have a surface normal that points more towards the source that generated the specular reflection than they actually should. Thus, reductions in specular reflections can improve imaging occurancy.

Photometric stereo imaging is based in the intensity variation due to illumination from different source positions. Intuitively, if the sources are moved closer together, there will be less intensity variation between images taken with different sources, and the signal to noise ratio in the surface normal estimation will decrease. To evaluate the performance of PSE with a light source separation and working distance used for endoscopic purposes, the 3D printed phantom with a known surface normal map was imaged using the modified endoscope of FIG. 5 at 10, 20, 30, and 40 mm frontal working distances. In general, PSE consistently estimated the morphology of ellipsoidal elevations and depressions with 1, 2.5, 5 and 10 mm height (and depth) in selected combinations of radiuses of 0.5, 1.25, 2.5 and 5 mm. In all estimations, the surface normal directions correctly show the elevation or depression as a region in which border surfaces are oriented outwards for elevations and inwards for depressions.

Noticeable artifacts present in these estimations include measurement noise, slope signal amplitude scaling, discretization of the curve, shape deformations, and albedo variations. The shape and albedo non uniformities may be caused by an uneven layer of paint, which was especially noticeable in the smaller radius features. The amplitude scaling of the estimated slope is dependent on the working distance. The discretization of the curve is noticeable in the smaller features and is also expected given the small portion of the FOV that they cover. For example, a 1 mm wide feature imaged at a 40 mm working distance covers only approximately 8 pixels across the images acquired with the modified endoscope.

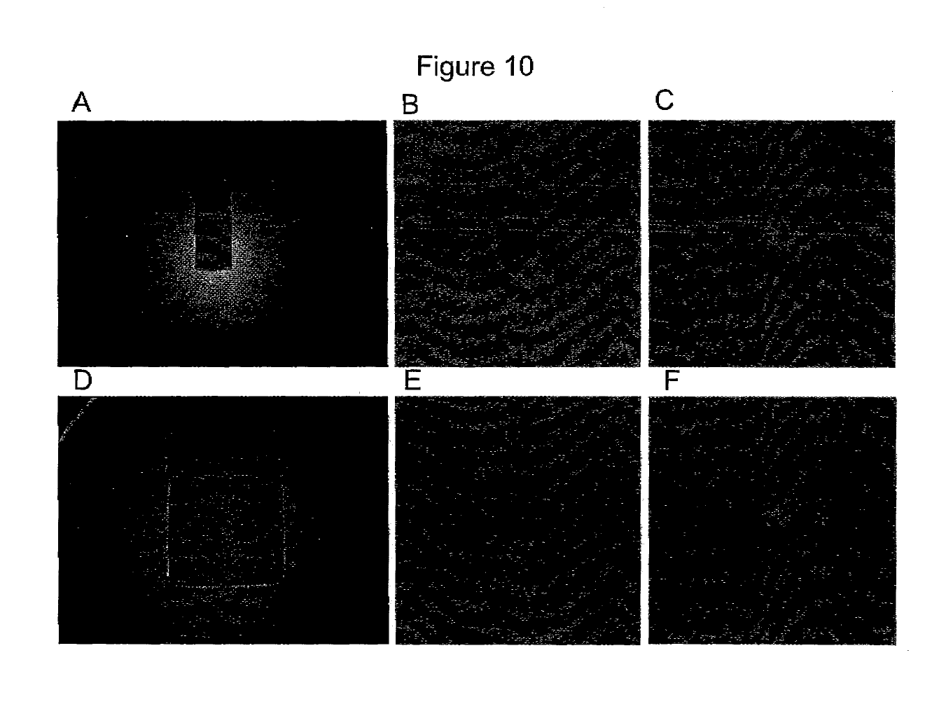

As an extreme example, FIGS. 10A-10F shows a 1 mm height, 0.5 mm radius bump imaged at 30 mm working distance. The conventional image in FIG. 10A is insufficient to discriminate the feature as an elevation or a depression, while its morphology is revealed in the surface orientations (10D) and the 3D rendering (10B). The surface orientations differ significantly from the numerical reference (10C), but maintain the gradient directions. An imperfection in the paint in the top of the elevation is imaged as a dark pixel in all the images in the series, appearing as a dark region in the conventional image and producing artifacts in the estimated morphology.

The results of the measurements demonstrate that PSE works in samples with complex geometries, including tubular environments. PSE is also able to reconstruct normal maps that are correlated to color images in ex-vivo human tissues with heterogenous optical properties. This demonstrates the power of the technique to separate a pixel's surface normal vector from its albedo. It is also observed that very fine folds (such as those present in FIG. 9c) are sometimes missed during reconstruction. These artifacts can be caused by deep, sharp folds in the tissue, where shadows are generated from multiple light sources, or by a poor signal to noise in the normal reconstruction. In both cases, a random error may be introduced to the resulting reconstructed normal and detailed topography can be lost. PSE can also suffer from artifacts resulting from specular reflection, e.g., wherein purely Lambertian remittance is assumed, additional reconstruction algorithms can actually use this specular information for more accurate normal map reconstructions. See, e.g., J. D. Waye, D. K. Rex, and C. B. Williams, Eds., Colonoscopy: Principles and Practice, 1st ed., Wiley-Blackwell (2003) the contents of which is incorporated herein by reference. Furthermore, implementing the technique with a higher resolution sensor, such as an HD endoscope, significantly increases the ability of PSE to capture fine topographical detail. Thus, preferred embodiments utilize imaging sensors with over 1 million pixels and preferably over 5 million pixels.

The measurements also demonstrate that PSE can accurately reconstruct normal maps from diminutive structures. The ability of PSE to reconstruct these normal maps is related to the difference in intensity that is registered for each pixel as it is illuminated from different light sources. Thus, if the light sources are moved closer together, the illumination of each pixel becomes more similar, and the normal reconstruction decreases in signal to noise. This is precisely what happens as the working distance is increased. It is observed that even with a low resolution image sensor, the signal to noise in the normal reconstruction can be sufficient to register topology changes from a 1 mm bump and depression at working distances of up to 40 mm. At this distance, the power from the light sources can limit the ability to image. The bulk of the screening for lesions is performed during the withdrawal of the endoscope, where the new field appears at the periphery of the image. Thus, in practice, the endoscopist is typically examining regions that are significantly closer than 40 mm from the endoscope tip.

The results demonstrated that, with appropriate changes, a commercial endoscope may be used to effectively implement PSE thereby providing new information on topography that is not present in conventional endoscopy. As shown in FIGS. 7-10, this topology can be visualized as normal maps or renderings, and other possible use models of the technique can be used. The additional information provided in the normal maps leads to better computer aided detection (CAD) algorithms for automatic lesion finding. PSE is also useful for improved mapping large regions of a sample (mosaicking), and generating novel morphology-based image enhancements (e.g. virtual chromoendoscopy). This technique has applications in polypectomy and laparoscopic surgery.

According to the present disclosure, an exemplary photometric stereo endoscope system may utilize highly miniaturized components, in where the light sources consist of highly efficient light emitting diodes (LED). These lights can be very small, are easy to control and synchronize electronically and they only require electrical connections from the control unit into the tip of the endoscope. This allows installing many illumination sources in the endoscope tip. Similarly, miniaturization of the detection electronics in the form of CCD or CMOS sensors allows covering a large total field of view by increasing the number of cameras installed in the tip of the endoscope instead of by designing a more complex lens system that covers a wide angle with a single detector array. In this configuration, the endoscope system has an advanced capability of leveraging the combination of information from multiple sensors and multiple illumination sources operated independently in synchronization. Thus, the topographical information acquired by combining series of pictures from each camera under different illumination conditions may be complemented with an enlarged field of view into a panoramic coverage of the endoscopy field of view. Multiple cameras that cover different fields of view with static illumination have been used to generate panoramic views in photography and endoscopy applications.

In further exemplary embodiments, an exemplary photometric stereo endoscope system may utilize multiple detectors with overlapping fields of view. This, configuration advantageously enables acquisition and reconstruction of low spatial frequency topographical information about the object, e.g., based on 3D imaging thereof from different viewpoints. According to the present disclosure, other means, such as focus or phase variation detection may also enable detection of low spatial frequency topographical information. Notably, in flexible endoscopy this capability may be limited in resolution by the lack of distinctive features in the tissues of interest, which need to be registered by software between the matching images to generate a three dimensional reconstruction. This limitation provides a lower resolution but with a quantitative value of distance measurement in the low spatial frequencies.

In exemplary embodiments, a low spatial frequency stereographic method for topography may be combined with the high spatial frequency photometric method for topography. This combination may enable quantitative measurement of the three dimensional surface shape, providing a further advantage as a method for measuring topography with multiple illumination sources and multiple detectors.

In some configurations multiple illumination sources and multiple detectors may be arranged to cover a sphere of vision around an endoscope head, e.g., by including detectors and/or illumination sources on both the distal tip as well as around the circumference of the endoscope head. In some embodiments, the endoscope head may include a circumferential arrangement of alternating detectors and light sources around the circumference of the endoscopic head, for example, in conjunction with a ring shaped arrangement of alternating detectors and light sources on the distal tip of the endoscopic head. In exemplary embodiments, the arrangement of the illumination sources and detectors, e.g., around the circumference and on the tip, may advantageously maximize source separation. In further embodiments, the arrangement of the detectors may provide for overlapping fields of view to enable a stereographic acquisition of topography information, e.g., in a forward-viewing portion of the endoscope field of view.

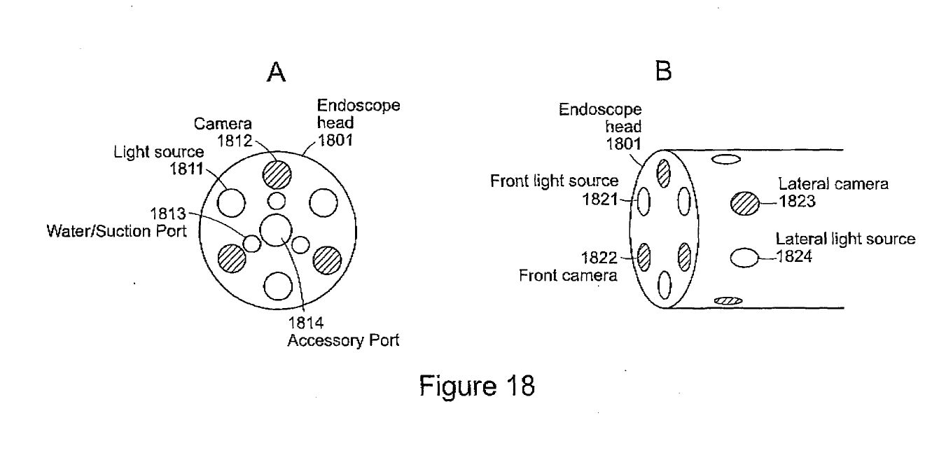

FIGS. 18A and 18B depict exemplary configurations of an endoscopic head for PSE, according to the present disclosure. More particularly, FIG. 18A depicts an exemplary endoscopic head 1801 including a ring arrangement of alternating light sources 1811 and detectors 1812 at a distal tip of the endoscopic head 1801. In the depicted embodiment, the endoscopic head includes three light sources and three light detectors. The endoscopic head may also include conventional endoscopic ports, e.g., accessory port 1814 and water/suction ports 1813. As depicted, each of the detectors 1812 may be associated with a water/suction port 1813, e.g., for cleaning the detector 1812 and maintaining a clean image. The accessory port 1814 may be used to introduce a tool or other accessory, e.g., for performing a resection, biopsy or the like. Advantageously, the PSE enabled systems of the present disclosure may enable real-time viewing of the tool or other accessory with PSE providing enhanced topography information about a sample being manipulated. FIG. 18B depicts a further exemplary configuration of an endoscopic head 1801 for PSE, according to the present disclosure. In particular, the endoscopic head 1801 of FIG. 18B includes both a ring arrangement of alternating front facing light sources 1821 and front facing detectors 1822 at a distal tip of the endoscopic head 1801 as well as a circumferential arrangement of alternating lateral facing light sources 1823 and lateral facing detectors 1824 around a circumference of the endoscopic head 1801. In the depicted embodiment, the endoscopic head includes three front facing light sources, three lateral facing light sources, three front facing detectors, and three lateral facing detectors. It will be appreciated that the number of light sources and detectors in the exemplary embodiments depicted in FIGS. 18A and 18B are note limiting.

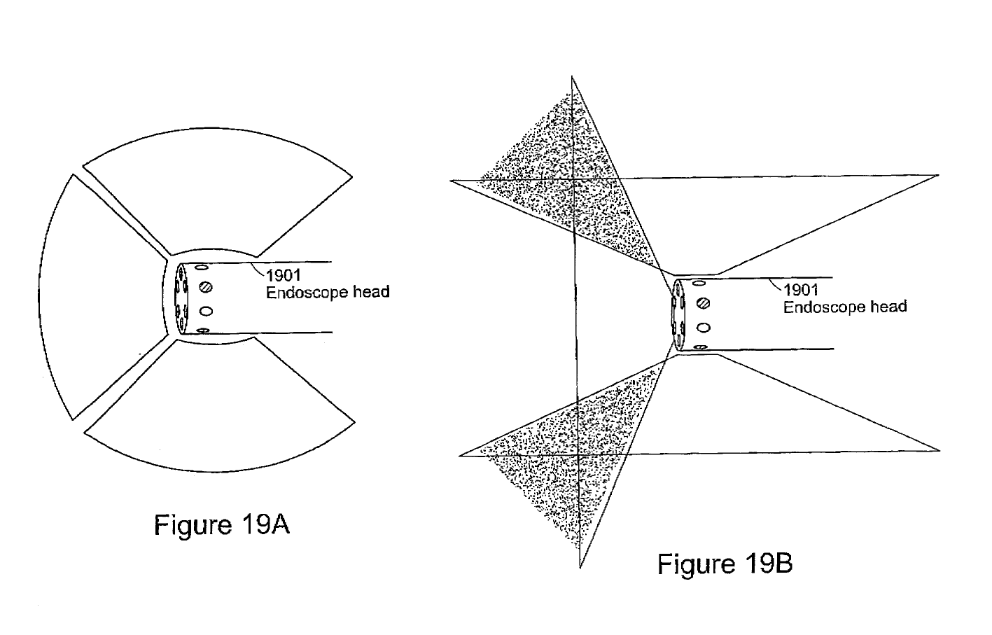

FIGS. 19A and 19B illustrate various advantageous of the configuration depicted n FIG. 18B. In particular, as illustrated in FIG. 19A the combination of lateral facing and front facing detectors advantageously enables imaging a sphere of vision around an endoscope head, e.g., similar to panoramic imaging. Moreover, the use of multiple detectors may advantageously enable using detectors with narrower fields of view than in conventional endoscopy, to achieve similar or larger field of view coverage. Furthermore, as depicted in FIG. 19B front facing and lateral facing cameras may include overlapping fields of view (shaded regions) thereby enabling stereographic acquisition of topography information. The use of front facing and lateral facing light sources may also enable greater source separation for higher PSE resolution.

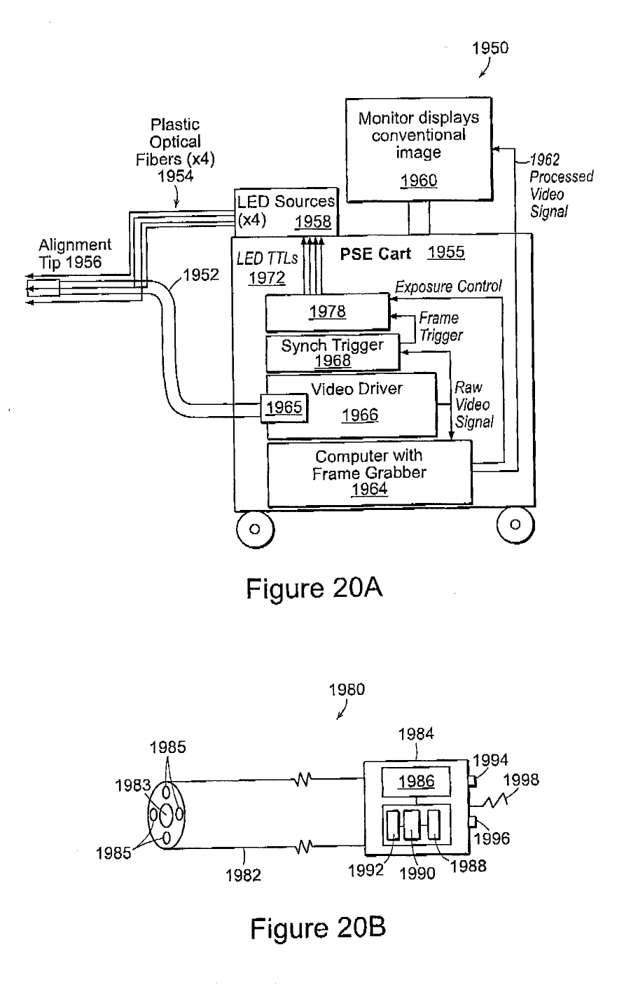

FIGS. 20A and 20B depicts an exemplary systems 1950 and 1980 capable of implementing PSE, according to the present disclosure. System 1950 in FIG. 20A may advantageously include a plurality of light sources 1958, e.g., LEDs, and a detector 1965, e.g. a CCD camera, operatively associated with a distal and 1952 of an endoscope, e.g., via optical fibers 1954. A light source controller 1970 and/or control logic 1972, such as transistor-transistor logic, may be used to control sequencing of the light sources 1958, e.g., in response to a frame rate single synched using synchronization logic 1968 from an image or video feed outputted from a video driver 1966 operatively associated with the detector 1965. A processor 1964, e.g., a computer, may receive the raw image or video feed from the video driver and process/analyze the signal, e.g., to implement PSE, virtual chromoendoscopy and/or CAD, such as described herein. The analyzed/processed signal 1962, including for example, processed image information and/or topographic information may be displayed using a monitor or other display device 1960. The processor 1964 may also be used to control the light sources 1958, e.g., to control the exposure thereof such as via the light source controller 1970. As depicted, system 1950 is a self-contained cart based system, e.g., a medical cart.

System 1980 in FIG. 20B may is advantageously depicted as a hand held system and may advantageously include a plurality of light sources 1985, e.g., LEDs, laser diodes, or the like and a detector 1983, e.g., a CCD camera, integrated into a distal end of an endoscope 1982. The hand held system may further include integrated system components such as a processor/power source 1992, memory 1990 and a communications system 1988, e.g., for communicating via a wireless transmitter and or cable 1998, as well as a control panel 1986 for including a user interface for controlling operation of the hand held system. Such integrated system components may advantageously be integrated, for example, directly into a handle 1984 for the endoscope 1982. System 1980 may also include one or more ports 1994 and 1996, e.g., for use as an accessory port or fluid/suction port.

In exemplary embodiment, PSE may be implemented in a self-contained imaging device that wirelessly transmits the image information to an external receiver. In particular, images of the field of view acquired by sequentially illuminating the object or using an illumination strategy described in this application can be transmitted by the self-contained device. The receiver can relay these images to a secondary processor, or have onboard processing to reconstruct the topographical information from these sequences of images. This self-contained imaging device can be swallowed or deposited in the colon by an endoscope, and then traverse the colon naturally or by mechanical means. The image sensors and illumination sources can be positioned on the tips of the pill to look forward and backward, and/or on the sides of the pill to view the colon wall laterally.

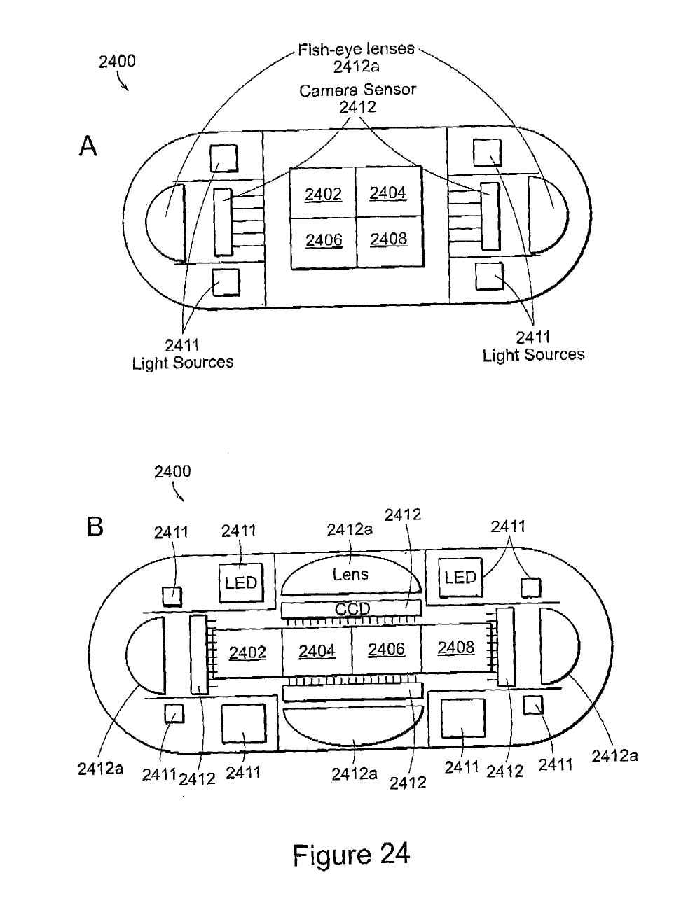

FIGS. 24A and 24B depict exemplary self-contained imaging devices 2400 for implementing PSE, according to the present disclosure. Imaging devices 2400 can include, for example, a plurality of light sources 2411, such as LEDs, and a plurality of image detectors, such as CCD cameras 2412. In the depicted embodiments, the plurality of image detectors 2412 may each include an associated optical system 2412a, e.g., a fish eye lens, for determining the field of view. The light sources 2411 and detectors 2412 can be positioned on the distal and proximal ends of the imaging device (as per the embodiments in both FIGS. 24A and 24B), e.g., to view forward and backward, and/or on the lateral sides of the imaging device (as per the embodiment in FIG. 24B), e.g., to view the colon wall laterally. Imaging devices 1400 may further include a processor/control logic 2402, memory 2404, a power source 2406 and a communication system 2408, such as a transmitter.

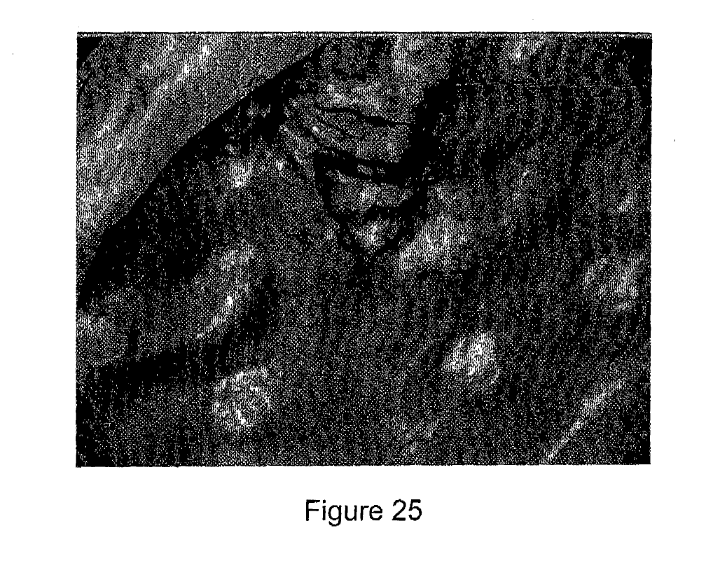

FIG. 25 depicts that topographic information acquired using the systems and methods described herein may be used to image surface texture and vasculature components as well as cript/pit patterns, and lesions. In particular, blood vessels appear as high contrast aspects in PSE. This demonstrates the high sensitivity and resolution possible using the systems and methods described herein. Resolution may be improved by using shorter wavelength light (e.g., U.V. light, that doesn't diffuse as easily, by decreasing the working distance (at the expense of the field of view), and/or by achieving greater source separation. In some embodiments, lower resolution imaging may be used to first identify possible lesions/features of interest and higher resolution may be utilized to analyze/classify the identified lesions/features. In some embodiments, high definition imaging may be used (e.g., greater than 1.5 MP to increase resolution. In some embodiments, high spatial frequency detection with PSE may be combined with a secondary imaging protocols such as low spatial frequency detection, e.g., using phase or focus variation measurements, stereoscopic imaging (e.g., 3D imaging) or the like. Such secondary imaging protocols may advantageously be implemented using overlapping hardware with the PSE system, e.g., shared detectors, light sources, etc.

As noted above, for the particular application of colorectal cancer screening by optical colonoscopy, an important limitation of current methods is that significant lesions are frequently missed due to poor contrast. One accepted way to increase lesion visibility is to spray a blue dye into the lumen to create color contrast at topographical changes in the mucosa (“chromoendoscopy”). However, because this technique is time-consuming, it is not used in routing screening. PSE can provide useful contrast to the endoscopist to increase lesion sensitivity of colonoscopy, without adding a significant increase to the colonoscopy procedure time, thus decreasing mortality rates from colorectal cancer. Unlike chromoendoscopy, PSE will not change the routine image that the endoscopist is used to seeing, and it will not significantly increase procedure time. Chromoendoscopy approximately doubles the time it takes to perform a colonoscopy, making it impractical for routine use. Unlike conventional colonoscopy, PSE is sensitive to inherent changes in surface topology that are commonly found from precancerous lesions.

With reference to FIG. 13B an exemplary algorithm 1300 for implementing virtual chromoendoscopy is depicted. According to the illustrated algorithm 1300, in exemplary embodiments, systems and methods for virtual chromoendoscopy may generally involve the following steps: 1310 acquire data that represents the topographical shape of the sample surface in an endoscopy setting 1320 optionally process the acquired dataset to simulate where a dye would accumulate and 1330 combine the information obtained from steps 1310 or 1320 with a co-registered conventional endoscopy image, e.g., overlaying the topographic information onto the image.

Table 4 enumerates several specific approaches to implementing each of these steps which may be used in any combination to embody the invention:

TABLE 4

| Step l | Step 2 (optional) | Step 3 |
|---|---|---|
| photometric stereo imaging | Curvature map | Overlay data from steps 1 |
| stereo imaging | Band-pass filter of | or 2 on conventional image |
| computed tomography | elevation changes | or vice-versa |
| colonoscopy (CTC) | Physical model of fluid | Use data from steps 1 or 2 |
| time-of-flight imaging | (e.g. Finite element model | as a mask for the filtering |
| plenoptic camera imaging | or Navier-Stokes fluid | the conventional image |
| LIDAR imaging | simulation). | (with various kinds of |
| Fourier profilometry | Texture or surface | filter, e.g. dodge, burn, |
| Phase imaging | roughness map | brightness, contrast, color, |
| Focus variation | | etc.) |
| Optical coherence | | Overlay data from steps 1 |
| tomography | | or 2 on a 3D rendering of |
| | | the conventional image. |

Notably, PSE is only one potential source for topographic imaging information, and the systems and methods related to virtual chromoendoscopy are not limited to systems and methods implementing PSE. PSE, however, provides a particularly elegant solution for simultaneously obtain both topographic and conventional image data using a same optical imaging system and image set. This, enables fast and easy data acquisition and processing, particularly as related to indexing and registering topographic information with respect to images.

FIG. 13C illustrates an example of the procedure and the type of image that can be generated using virtual chromoendoscopy. Using photometric stereo endoscopy, once can simultaneously obtain a conventional endoscopic image (13C) and a topographical map (13D) of the sample. The data from the topographical map can then be processed to simulate where a dye would accumulate if it were sprayed on the sample. Combining the processed topographical information with the conventional endoscopic image results in an image which looks similar to that obtained with chromoendoscopy (13F).

In comparison with conventional chromoendoscopy PSE, as described herein, advantageously enables acquisition of topology information without the need to spray and rinse a dye. Such topology information obtained from the PSE may be advantageously overlayed or otherwise combined with conventional imaging data, for example, 2D or 3D imaging data, to produce an image that resembles a chromoendoscopy type image without the need to spray, inject, or otherwise apply a physical dye. As used herein such image augmentation may be referred to as Virtual Chromoendoscopy Augmented by Topology (VCAT).

With reference to FIG. 17 an exemplary algorithm 1700 for implementing VCAT is depicted. According to the illustrated algorithm 1700, in exemplary embodiments, systems and methods for virtual chromoendoscopy may generally involve the following steps: 1710 acquire data that represents both the image and the topographical shape of the sample surface in an endoscopy setting 1720 extract features from both the image and the topology information, for example features related to legions, blood vessels, surface texture, pit patterns, curvature of the surface, three dimensional orientation of the surface and the like, and 1730 combine such features to produce an image augmented by topology information, for example for guiding the attention of an endoscopist towards changes in topology. In exemplary embodiments, the augmented image may include a color overlay over a conventional (for example, 2D or 3D), the overlay highlighting changes in topology (for example, simulating a chromoendoscopy dye), or highlighting/classifying topographical features in the image, such as legions, blood vessels, surface texture, pit patterns, curvature of the surface, three dimensional orientation of the surface and the like. In some embodiments, the creation of the augmented VCAT image may include receiving a selection of one or more topographical features for overlaying over a conventional image. In some embodiments, the selected topographical features as well as characteristics of the overlay such color and transparency, may be dynamically adjusted when viewing the augmented VCAT image.

In exemplary embodiments, various imaging techniques may be used to obtain topography information, for augmenting conventional image data. Such techniques may include but are not limited to PSE as described herein. Table 6, below, includes a list of imaging techniques which may be used for obtaining topology information per step 1710 of FIG. 17, a list of features which may be extracted from each of the image and topology information per step 1720 of FIG. 17, and a list of algorithms for combining such extracted features into an augmented image, per step 1730 of FIG. 17.

TABLE 6

| Means to obtain | Features extracted from the | Feature combination |
|---|---|---|
| topological information | image and the topology | algorithms |
| photometric stereo imaging | Image Features: | Linear combination |
| stereo imaging | Image luminance | Region-based combination |
| computed tomography | Image chrominance | (i.e. segmentation of |
| colonoscopy (CTC) | Image gradient | structures and then |
| time-of-flight imaging | Second-order derivatives | combining features |
| plenoptic camera imaging | based measurements | differently in each of the |
| LIDAR imaging | Ridge detection | segmentations) |

| | | |
|---|---|---|
| Fourier profilometry | Frangi | Machine-learned based |
| Phase imaging | Sato, . . . | combinations |

| | | |
|---|---|---|
| Focus variation | Blob detection | Physical model of fluid |

| | | |
|---|---|---|
| Optical coherence | Laplacian of Gaussian | (e.g. Finite element model |

| | | |
|---|---|---|
| tomography | Optical flow, . . . | or Navier-Stokes fluid |
| Gradient based images | Topography Features: | simulation). |
| Hard wavelet imaging | Valleys | |
| | Ridges | |
| | Normal vector orientation | |
| | Normal vector divergence | |
| | Normal vector gradient | |
| | Ridge detection on the | |
| | topology map | |
| | Blob detection on the | |
| | topology map | |
| | Curvature map, . . . | |

A person skilled in the art will understand that several of the features mentioned in Tables 4 and 6 can be computed at different image scales. Thus, the extracted features may be referenced in a scale-space domain. Moreover, while the algorithms described herein for combining imaging information and the topographical information may be applied at a particular time, e.g., time stamp n, nothing prevents the same paradigm from being extended to more temporal steps or to a recursive algorithm. Thus, for example, features may be extracted and analyzed at frames n, n−1, n−2, . . . or any combination thereof. This may be relevant when analyzing features based on movement, such as optical flow.

In some embodiments, the combination of the extracted features may be achieved using in a machine-learning paradigm. In particular, topology information (for example, topographical map information) and image information (for example, 2D or 3D image information, may be acquired, for example, from regions of the patient where chromoendoscopy is typically performed. The acquired information including topology information and image information may constitute a training dataset based on features extracted therefrom. In particular, extracted features from the topology and image information may be used as the parameters for the machine-learning paradigm, for example, whereby a function is learned/trained to combine the features in a desired manner, for example, so as to best resemble conventional chromoendoscopy imaging. Examples of learned/trained functions are: linear combination, support vector machines, decision trees, etc. Resemblance between virtual chromoendoscopy images and conventional chromoendoscopy images can be measured as Root Mean Squared Error (RMSE) or more advanced metrics such as the Structural Similarity Index (SSIM). Such function may then used to produce the virtual chromoendoscopy images. Machine learning may also be used to identify which features combinations, may best be used in CAD (computer aided detection) and computer aided classification of lesions or other physiological characteristics relevant to treatment or diagnosis. Thus, a VCAT image may be automatically tailored specifically for detection/identification of particular/selected physiological characteristic(s).

According to exemplary embodiments of the disclosure, the following exemplary VCAT algorithm may be implemented:

Input: PSE image I. Weight vector w.
Output: VCAT image I_{VCAT}
Algorithm: for new image Iₙ do
Remove specular reflections to generate I^{c}ₙ
Compute the normal map and height map using PSE
Estimate a uniformly illuminated image from the PSE images: Iᵤ
Equalize the image to match the color an intensity properties of a canonical chromoendoscopy image: Iₑ
Combine the height, the normal map and Iₑ according to the weight vector w to generate the luminance I^{L}_{VCAT}
Combine the luminance information with the chrominance information of Iₑ to generate I_{VCAT}

Note that the weight vector w may be computed by minimizing the RMSE on a training dataset.

In one exemplary embodiment of topographic virtual chromoendoscopy, a photometric stereo endoscopic imaging system including of multiple illumination sources and or multiple detectors may be used to acquire topographical information of a sample. From the obtained topographic information, metrics may be computed to represent the texture and surface roughness of the sample; the arrangement, density and orientation of pits and crevasses in the sample; or the gradients and curvature tensor of the object surface. These metrics may be combined into a channel that represents a parameter of interest, such as the signed curvature of the surface in each image pixel. This parametric channel is applied may then be applied a filter or function to the standard 2D color image of the sample, changing the hue of image from red to dark blue using a lookup table that maps the parametric channel into a visible dark blue accent in the color image corresponding to the surface property, such as mapping the curvature to the color image to proportionally enhance with a blue color the amount of curvature in the corresponding surface region.

In another exemplary embodiment of topographic virtual chromoendoscopy, a plenoptic camera imaging system may be used to obtain topographical information about the sample. The plenoptic camera system may include a CCD/CMOS imaging sensor, a principal optical system comprised by one or more lenses to focus light from the object area of interest into the sensor, and a secondary optic system comprised by a lenslet array that transforms each region in the image plane into a focused sub-region or macropixel in the sensor image plane. The plenoptic camera is capable of using a single high resolution sensor to acquire multiple lower resolution images that have different effective viewpoints. With multiple images of the endoscopic sample acquired from different effective viewpoints, even under a single lighting condition, a three-dimensional reconstruction of the sample surface may be computed by identifying corresponding features in images from different orientations, and calculating the geometric position with respect to each viewpoint position and the position of the features within the images. Notably, if few distinctive features can be matched between the corresponding images, computation may result in a low spatial resolution three dimensional orientation.

Furthermore, a plenoptic camera, in combination with one or more synchronized illumination sources, can obtain and compute topographical information about an object area for real time applications. From this surface topography, the surface shape is refined with a selective spatial frequency filter to correct for artifacts, and the deposition of a physical fluid is simulated by considering the surface shape and mechanic properties of the surface and the fluid. The color image, together with the surface shape and the simulated fluid are displayed in a three dimensional rendering of the object.

In yet another exemplary embodiment of topographic virtual chromoendoscopy, an optical coherence tomography endoscopic system may be used to acquire topographical information of the sample, The optical coherence tomography system may include a coherent laser illumination source and an interferometer that correlates the light scattered by the object with the reference illumination, and one or more detectors that record the amplitude of the correlation resulting from the interferometer. The imaging system allows reconstructing three dimensional images of the tissue microarchitecture and topography up to light penetrating depth, including the surface shape, different tissue surface layers and blood vessels. This imaging method is suitable for real time applications. Using the topography acquired in this way, the size, location, depth, orientation, and arrangement of blood vessels in the tissue surface is analyzed to identify abnormal patterns. The information is displayed to the endoscopist in the form of a two dimensional color image with an overlaid marker that indicated the location of an area that has been identified as having an abnormal pattern of tissue microarchitecture. This marker can be an arrow, a circle, or other predefined marker that does not interfere with the regular use of the color image.

Measurements were conducted on the virtual chromoendoscopy techniques described herein. Videos of tissue illuminated from a sequence of four alternating white-light sources were acquired with a modified Pentax EG-2990i gastroscope. A Pentax EPK-i5010 video processor which outputs a digital signal that is synchronized with the 15 Hz frame rate of the endoscope image sensor. The synchronization pulses were converted to a cycle of four sequential pulse trains that were sent to an LED driver via an Arduino microcontroller [12]. The LEDs were coupled to light guides with diffusing tips at the distal end. The conventional light sources were turned off and only the custom LED sources were used to illuminate the sample. The four optical fibers were oriented at equal angles about the center of the gastroscope tip. The resulting system acquired high-definition images (1230×971 pixels) and enabled topographical reconstructions every four frames (3.75 Hz) in a system that has the same outer diameter (14 mm) as conventionally-used colonoscopes.

The high frequency topography of the field of view was calculated using a photometric stereo endoscopy method which reduces errors arising from an unknown working distance by assuming constant source vector directions and high-pass filtering the calculated topography map (11). The underlying assumption is that the error incurred in the fixed estimation of light source positions changes slowly from pixel to pixel, and can thus be corrected by filtering the shape gradients with a spatial frequency high-pass filter. The four source vectors for all pixels in the image were assumed to be equal to that of a pixel in the center of the field-of-view, for which source vectors were calculated assuming a 40 mm working distance. The resulting x and y gradients calculated by photometric stereo were high-pass filtered by subtracting a low-pass image resulting from blurring gradients with a pixel Gaussian kernel with σ=100 pixels. A height map was estimated from the high-pass filtered gradients using a multigrid solver for the Poisson equation that minimizes integration errors (11).

To demonstrate and validate the potential of topography-based virtual chromoendoscopy, the same field of view of ex-vivo swine colon was imaged before and after applying a chromoendoscopy dye. The swine colon was cleaned, cut, and spread open on a surface. The PSE endoscope was fixed above the tissue and images were acquired before and after spraying and rinsing an approximately 0.5% solution of indigo carmine chromoendoscopy dye. To achieve virtual chromoendoscopy augmented with topography, PSE was used to simultaneously acquire conventional white light images and topography information. Specifically, the uniformly illuminated image Iᵤ, the surface normal maps N, and the tissue height maps h from the PSE images were calculated. VCAT combined information from the conventional, uniformly-illuminated image and topographical measurement to emulate the dye accumulation in topographical features in dye-based chromoendoscopy.

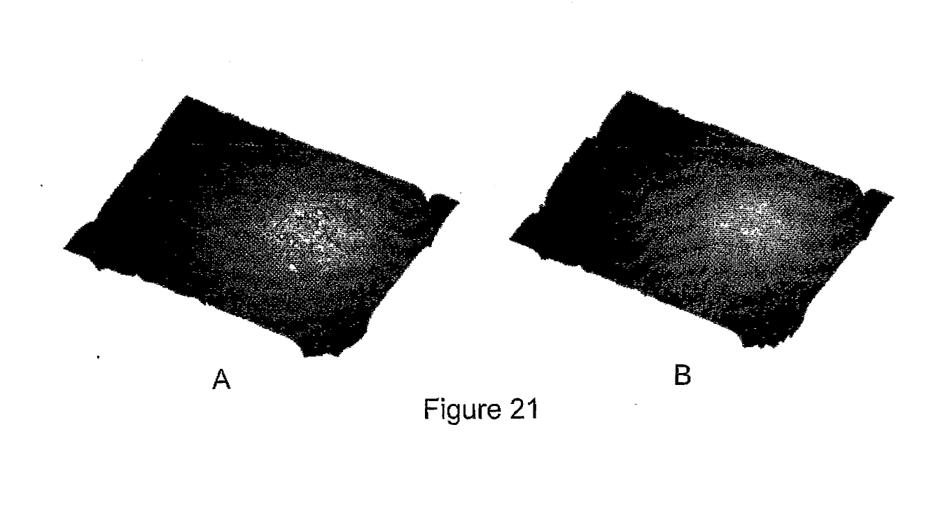

In the measurements conducted, a photometric stereo algorithm was used that assumed that the object had a Lambertian surface remittance. Consequently, specular reflections from the wet tissue surface created artifacts in the topographical reconstruction. These errors created artificial dips and bumps that may be highlighted by virtual chromoendoscopy. Since the fiber optics of the photometric stereo system closely represent point sources and the surface of the colon varies smoothly, specular reflections appeared in the images as circular-like shapes of high brightness. To detect such specular reflection a scale-space approach based on the Laplacian of a Gaussian filter was used. In particular, for each image Iₙ, its convolution with a Laplacian of Gaussian filters at different scales σ was computed and normalized them with σ². The scale-space approach was then projected into a single 2-dimensional image I_{L}=max_{σ}(Iₙ*σLoG_{σ})σ². Pixels that had a greater value than the mean plus three standard deviations in I_{L} were considered specular reflections and were removed from the image. The values of the corrected image Iₙ^{c} at those locations were estimated by solving Laplace's equations from its boundary pixels. FIG. 21 depicts the surfaces reconstructed by PSE before (a) and after (b) removing specular reflections.

For each set of images I, features were computed that were combined to generate a virtual chromoendoscopy luminance image. In the measurements conducted, the following features were computed that were based on both the image information as well as the topography:
Equalized uniformly illuminated image Iₑ: Iₑ was computed as the L channel of the mean value of the four sequential images acquired by the PSE system, after correcting for specular reflections and converting into Lab color space. Ī=(Iₙ^{c}+Iₙ₋₁^{c}+Iₙ₋₂^{c}+Iₙ₋₃^{c})/4. The brightness and contrast of the uniformly illuminated image were adjusted so that it matches those of a conventional chromoendoscopy image.
Height map: The height map obtained from PSE was decomposed into two features: pits and crevices, depending on whether the height map was positive or negative.
Angle of the surface normal (θ): The angle of the surface normal was computed with respect to the z direction.
Image offset: A vector of ones added to compensate for image offsets.

One possible goal of virtual chromoendoscopy is to replicate as faithfully as possible a conventional chromoendoscopy image I_{ch}. To that end, the composition of a VCAT image may be framed as a minimization problem where the features f are linearly combined and the cost function is the mean square error when compared relative to the conventional chromoendoscopy image I_{ch}. Thus, in measurements conducted, problem was defined as finding the set of weights that minimize:

$$\hat{w} = {arg min}_{w} ‖ {I}_{ch} - f \cdot w ‖ / {N}_{pix} .$$

This linear problem may be solved by applying Moose-Penrose pseudoinversion of the feature matrix and multiplying it by the objective image:

\hat{ω}=pinv(f)·I_{ch}.

The same process can be applied when estimating the weighting vector \hat{ω} with several images by changing the conventional image I_{ch} and the features f for a concatenation of the images and features.

Given an input image Iₙ and its features f, a luminance component of the virtual chromoendoscopy image may be estimated as a linear combination of the features, using as weights \hat{ω}:

$${I}_{VCAT}^{L} = {\sum }_{i = 1}^{5} {\hat{w}}_{i} {f}_{i} .$$

The color components of the virtual chromoendoscopy image may be obtained by equalizing the chrominance of the original image Iₙ to match the chrominance of the conventional chromoendoscopy image.

Measurements conducted used exemplary Algorithm 1, below, for VCAT:

Algorithm 1: Virtual chromoendoscopy
augmented with topography

Data: Photometric Stereo Images I. Weight vector w
Result: Virtual Chromoendoscopy Image I_{VCAT}
while New Image Iₙ do

| | | |
|---|---|---|
| \| | - | remove specular reflections from Iₙ → Iₙ^{c} |
| \| | - | perform PSE to compute the normal and height maps |
| \| | | I^{c} = {Iₙ^{c}, Iₙ₋₁^{c}, Iₙ₋₂^{c}, Iₙ₋₃^{c}} → {h, N} |
| \| | - | estimate a uniformly illuminated image from I^{c} → Iᵤ |
| \| | - | equalize the image to match the color and intensity properties of a |
| \| | | canon chromoendoscopy image Iᵤ → Iₑ |
| \| | - | generate features from {Iₑ, h, N } → f |
| \| | - | combine the features to generate the VCAT image I_{VCAT} = f(f) |

end

In measurements conducted, the brightness and contrast of each I_{ch} acquired were equalized in three different swine colons to reduce illumination artifacts, which was defined as {I_{ch}ⁱ}. The VCAT images were evaluated by comparing I_{VCAT}ⁱ with I_{ch}ⁱ.

Leave-one-out cross-validation was used to estimate the performance of the system on unseen images. For each image sample i, the weighting vector \hat{ω} was computed with the remaining pair of PSE images and conventional chromoendoscopy and the estimated I_{VCAT}ⁱ was reconstructed. In order to evaluate if the topographical features f noted herein facilitate the generation of realistic VCAT images, virtual chromoendoscopy images were generated without using the topographical features: I_{ch}ⁱ. This was accomplished by adjusting the brightness, contrast and color channels of the uniformly illuminated images to that of the conventional chromoendoscopy image. The two sets of virtual chromoendoscopy images, {I_{VCAT}ⁱ} and {I_{vc}ⁱ} were compared to the objective image, {I_{ch}ⁱ}, using two similarity measurements: root mean squared error (RMSE) and the structural similarity index (SSIM). See, e.g., Wang, Z., Bovik, A., Sheikh, H., Simoncelli, E.: Image quality assessment: from error visibility to structural similarity. IEEE Transactions on Image Processing 13(4) (April 2004) 600-612. The SSIM index is a framework for image comparison as a function of their luminance, contrast, and structural similarity. Since the norm of the difference between {I_{VCAT}ⁱ} and {I_{ch}ⁱ}, was minimized, the RMSE correspondingly decreased within the training set. Given, that leave-one-out cross-validation was utilized, the RMSE from the test image is a valid metric for evaluation.

Measurements also demonstrated the proposed VCAT techniques using three different videos of the porcine colon. However, the effects of virtual chromoendoscopy in such videos were not quantified since corresponding registered conventional chromoendoscopy frames were not available for comparison.

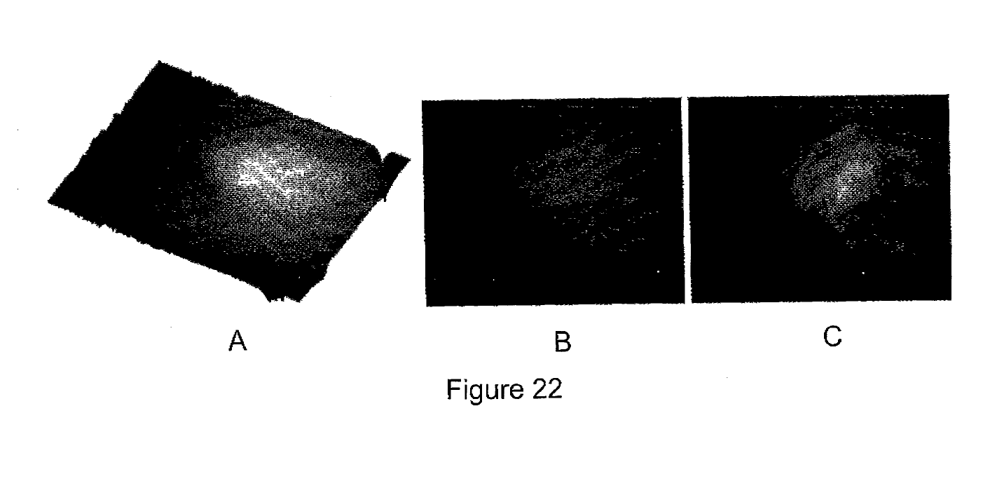

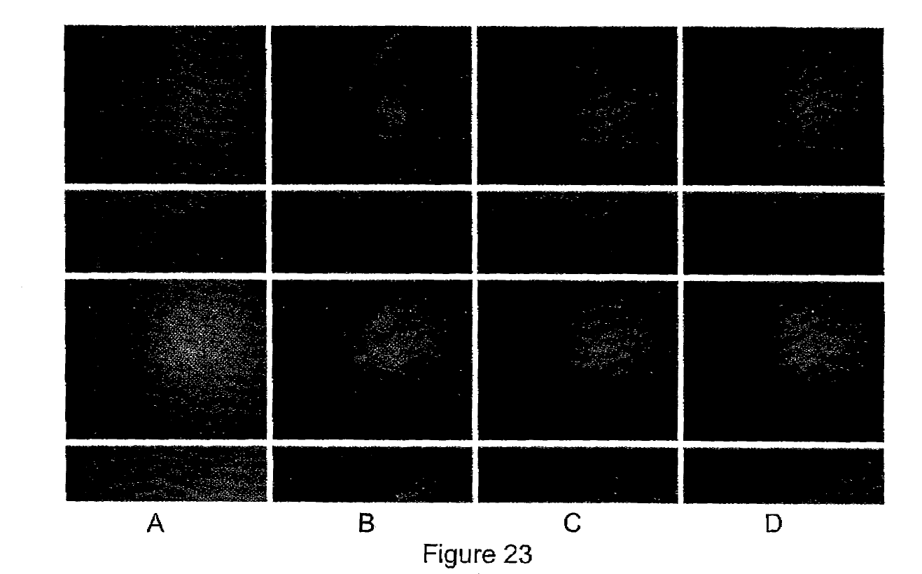

FIGS. 22 and 23 each compare images obtained from VCAT and conventional chromoendoscopy. As expected, images from VCAT incorporate topographical contrast by highlighting the ridges and darkening the pits in the colon mucosa. FIG. 23 also shows virtual chromoendoscopy obtained by color equalization. Qualitatively, VCAT produces images that are more similar to conventional chromoendoscopy than virtual chromoendoscopy by color equalization.

More particularly, in FIG. 22 depicts (a) topography obtained by PSE; (b) virtual chromoendoscopy calculated by incorporating features from the PSE obtained topography with respect to a conventional (non-dyed) image in the same field of view; and (c) Dye-based chromoendoscopy image performed in the same field of view.

FIG. 23 depicts, for two different samples of training images (rows 1 and 2 and rows 3 and 4, respectively) used (note that the second and fourth rows depict zoomed in regions of the samples depicted in the first and third rows, respectively) each of (a) original images after removing specular reflections; (b) image of same field of view as (a) applying conventional dye-based chromoendoscopy; (c) corresponding VCAT images; and (d) virtual chromoendoscopy by equalizing the color statistics of the conventional image in (a) column to that of the chromoendoscopy image in (b). Qualitatively, the VCAT technique appears to enhance regions with ridges in the same way that conventional chromoendoscopy does and demonstrates an improvement over virtual chromoendoscopy by equalizing the color statistics

The quantification of the image improvement is shown in Table A:

TABLE A

| | Measure | | | | | | | |
|---|---|---|---|---|---|---|---|---|
| | RMSE | | | | SSIM | | | |
| Sample | S1 | S2 | S3 | p-value | S1 | S2 | S3 | p-value |
| I_{VCAT} | 3.06 | 6.32 | 4.12 | 0.08 | 0.882 | 0.747 | 0.848 | 0.026 |
| I_{vc} | 3.21 | 6.68 | 4.28 | | 0.815 | 0.704 | 0.771 | |

Table A, demonstrates quantification of the similarity between conventional chromoendoscopy and each of the proposed virtual chromoendoscopy (VCAT) and virtual chromoendoscopy by color equalization (vc) for the two evaluation metrics: RMSE and SSIM. Notably, incorporating topographical features results in both lower RMSE and higher SSIM. A student t-test was also performed on the results to show their statistical significance. Although only three points were used in the dataset, the improved p-value for the SSIM metric is statistically significant.

While PSE can reconstruct the 3D topography of the colon surface, the interpretation of this additional information may require a steep learning curve for a gastroenterologist. Chromoendoscopy, on the other hand, highlights features from the colon topography in a way that is intuitive and familiar to gastroenterologists. The measurements conducted confirm that VCAT can be used to generate images that are similar to conventional chromoendoscopy but incorporate the 3D topography for the field (for example, utilizing PSE as described herein).

Another field that stands to benefit from the capability of PSE to simultaneously acquire both conventional endoscopic imaging information and topographic imaging information is computer aided detection (CAD). In particular, systems and methods are disclosed herein which utilize new computer aided detection (CAD) algorithms to detect features in an endoscopy setting based on both conventional parameters (such as optical intensity patterns) and topographic parameters (such as derived using PSE).

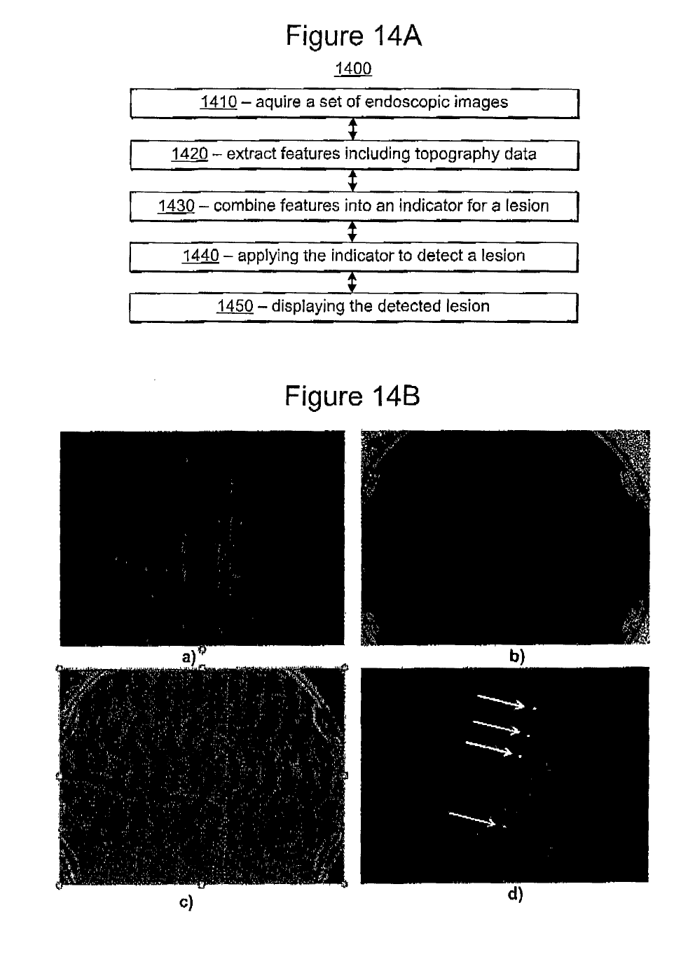

With reference to FIG. 14a an exemplary algorithm 1400 for implementing CAD using a PSE system is depicted. According to the illustrated algorithm 1400, in exemplary embodiments, systems and methods may implement computer aided detection of colon lesions by the following steps or a subset thereof:
1410 Acquiring at least one image from the video stream of the colonoscope.
1420 Extracting a set of features from such image or images. Such features are based on the image information and the topology of the colon under analysis.
1430 Combining such features into an indicator that measures the likelihood that a lesion is present in a given location.
1440 Making a decision on whether a lesion is present or not in a location based on the value of such indicator in at least the location under analysis.
1450 Displaying the decision to the doctor in the screen of the colonoscopy system.

Table 5 enumerates several specific approaches for implementing each of these steps. The present disclosure is not limited to any particular combination or combinations of the noted approaches:

TABLE 5

| Step 2: | | Step 3: | Step 4: | Step 5: |
|---|---|---|---|---|
| Features | | Algorithm | Decision | Information |
| Color | | Rule based | Non-maxima | Displaying |
| Contrast | | systems. | suppression. | a marker to |
| Image operators | | Linear | Thresholding. | the doctor. |
| | Vesselness. | combination. | Hysteresis | Changing the |
| | Sobel edges. | Support Vector | thresholding. | color of the |

| | | |
|---|---|---|
| Topology operators | Machines. | surface. |

| | |
|---|---|
| Curvature. | Neural Networks. |
| Orientation of | Decision trees. |
| the normal of | Random forests. |
| the surface. | Boosting. |
| Divergence of | Gaussian process |
| the surface | regression. |
| normal. | ADAboost |

FIG. 14b illustrate example imaging data obtained using PSE and the advantageous results of using results of applying an exemplary CAD algorithm based on such imaging data. More particularly, (a) depicts a conventional mage obtained with a colonoscope; (b) illustrates the magnitude of the topological gradient obtained via PSE with the colonoscope; (c) depicts the conventional image filtered with a laplacian of gaussian filter to enhance protuberances; (d) depicts the result of our the application of a CAD algorithm that combines the topological information of image (b) and the filtered image of (c). The automatically detected lesions are highlighted with arrows.

Table 7 further enumerates exemplary approaches for implementing CAD, e.g., via an algorithm, such as algorithm 1400 of FIG. 14a. The present disclosure is not limited to any particular combination or combinations of the noted approaches:

TABLE 7

| Step 2: | | Step 3: | Step 4: | Step 5: |
|---|---|---|---|---|
| Features | | Algorithm | Decision | Information |
| Image Features: | | . Bayesian | Non-maxima | Displaying |
| Image luminance | | classifiers | suppression. | a marker to |
| Image chrominance | | Gaussian processes | Thresholding. | the doctor. |
| Image gradient | | Isotonic regression | Hysteresis | Changing the |
| Second-order | | Support vector | thresholding. | color of the |
| derivatives based | | machines | | surface. |
| measurements | | Multilayer | | |
| Ridge detection | | perceptrons | | |
| Frangi | | ADTrees | | |
| Sato, . . . | | J48 Trees | | |
| Blob detection | | M5P | | |
| Laplacian of | | Kstar | | |
| Gaussian, . . . | | Decision tables | | |
| . . . | | M5Rules | | |
| Topography Features: | | Random forests | | |
| Pits | (height < 0) | AdaBoost with haar | | |
| Crevices | (height > 0) | wavelets | | |

| | |
|---|---|
| Normal vector | Bagging |
| orientation | LogitBoost |
| Normal vector | Voting . . . |
| divergence | |
| Normal vector | |
| gradient | |
| Ridge detection on | |
| the topology map | |
| Blob detection on | |
| the topology map | |
| Curvature map . . . | |
| . | |

One embodiment of a CAD technique, according to the present disclosure, is as follows:
1. Obtain image and topography data using PSE: I(x)
2. Label such data by placing a bounding box around each visible lesion on the images, thus creating a training dataset.
3. For each image and for each topology map create a set of features based on their sobel gradients: gₒ(I(x))
4. Use such gradients to discern between lesions and regular tissue using haar wavelets and an AdaBoost algorithm. Haar wavelets are difference of integrals of the features on the surroundings of an image location. AdaBoost selects the set of Haar wavelets that optimally discern between lesion and not, as well as the set of weights that optimally combine such wavelets and a set of thresholds over such wavelets by minimizing the empirical error on a training dataset. More precisely, AdaBoost learns the function:

$$f ( x ) = {\sum }_{t = 0}^{T} {α}_{t} {h}_{t} ( x ) ,$$

where hₜ(x) is a weak classifier and corresponds to:

$${h}_{t} ( x ) = \{ \begin{matrix}- 1 & if {\int }_{{a}_{1}}^{ } {g}_{o} ( I ( x ) ) d x - {\int }_{{a}_{2}}^{ } {g}_{o} ( I ( x ) ) d x > thr \\ - 1 & otherwise\end{matrix}$$

5. Use f(x) that function to detect lesions in unseen images.

It is explicitly contemplated that the systems and methods presented herein may include one or more programmable processing units having associated therewith executable instructions held on one or more computer readable medium, RAM, ROM, hard drive, and/or hardware. In exemplary embodiments, the hardware, firmware and/or executable code may be provided, for example, as upgrade module(s) for use in conjunction with existing infrastructure (for example, existing devices/processing units). Hardware may, for example, include components and/or logic circuitry for executing the embodiments taught herein as a computing process, e.g. for controlling one or more light sources.

Displays and/or other feedback means may also be included to convey calculated/processed data, for example topographic information such as derived using PSE. The display and/or other feedback means may be stand-alone or may be included as one or more components/modules of the processing unit(s). In exemplary embodiments, the display and/or other feedback means may be used to visualize derived topographic imaging information overlaid with respect to a conventional two-dimensional endoscopic image, as described herein. In other embodiments the display and/or other feedback means may be used to visualize a simulated dye or stain based on the derived topographic imaging information overlaid with respect to a conventional two-dimensional endoscopic image. In exemplary embodiments, the display may be a three-dimensional display to facilitate visualizing imaging information.

The actual software code or control hardware which may be used to implement some of the present embodiments is not intended to limit the scope of such embodiments. For example, certain aspects of the embodiments described herein may be implemented in code using any suitable programming language type such as, for example, assembly code, C, C# or C++ using, for example, conventional or object-oriented programming techniques. Such code is stored or held on any type of suitable non-transitory computer-readable medium or media such as, for example, a magnetic or optical storage medium.

As used herein, a “processor,” “processing unit,” “computer” or “computer system” may be, for example, a wireless or wire line variety of a microcomputer, minicomputer, server, mainframe, laptop, personal data assistant (PDA), wireless e-mail device (for example, “BlackBerry,” “Android” or “Apple,” trade-designated devices), cellular phone, pager, processor, fax machine, scanner, or any other programmable device configured to transmit and receive data over a network. Computer systems disclosed herein may include memory for storing certain software applications used in obtaining, processing and communicating data. It can be appreciated that such memory may be internal or external to the disclosed embodiments. The memory may also include non-transitory storage medium for storing software, including a hard disk, an optical disk, floppy disk, ROM (read only memory), RAM (random access memory), PROM (programmable ROM), EEPROM (electrically erasable PROM), flash memory storage devices, or the like.

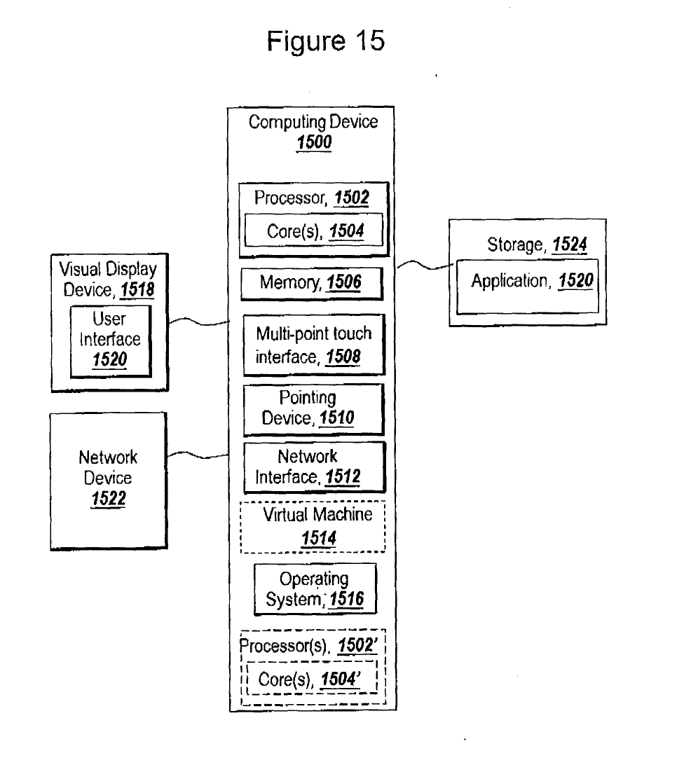

FIG. 15 depicts a block diagram representing an exemplary computing device 1500 that may be used for processing imaging information as described herein, for example to implement a PSE system. In particular, computing device 1500 may be used for processing imaging information from the imaging device for the plurality of different lighting conditions to calculate topographic information for the target surface, wherein the calculated topographic information emphasizes high frequency spectral components. The computing device 1500 may be any computer system, such as a workstation, desktop computer, server, laptop, handheld computer, tablet computer (e.g., the iPad™ tablet computer), mobile computing or communication device (e.g., the iPhone™ mobile communication device, the Android™ mobile communication device, and the like), or other form of computing or telecommunications device that is capable of communication and that has sufficient processor power and memory capacity to perform the operations described herein. In some embodiments, e.g., for CAD, a distributed computational system may be provided comprising a plurality of such computing devices.

The computing device 1500 includes one or more non-transitory computer-readable media having encoded thereon one or more computer-executable instructions or software for implementing exemplary methods and algorithms as described herein. The non-transitory computer-readable media may include, but are not limited to, one or more types of hardware memory, non-transitory tangible media (for example, one or more magnetic storage disks, one or more optical disks, one or more USB flash drives), and the like. For example, memory 1506 included in the computing device 1500 may store computer-readable and computer-executable instructions or software for implementing exemplary embodiments. The computing device 1500 also includes processor 1502 and associated core 1504, and in some embodiments, one or more additional processor(s) 1502′ and associated core(s) 1504′ (for example, in the case of computer systems having multiple processors/cores), for executing computer-readable and computer-executable instructions or software stored in the memory 1506 and other programs for controlling system hardware. Processor 1502 and processor(s) 1502′ may each be a single core processor or multiple core (1504 and 1504′) processor.

Virtualization may be employed in the computing device 1500 so that infrastructure and resources in the computing device may be shared dynamically. A virtual machine 1514 may be provided to handle a process running on multiple processors so that the process appears to be using only one computing resource rather than multiple computing resources. Multiple virtual machines may also be used with one processor.

Memory 1506 may include a computer system memory or random access memory, such as DRAM, SRAM, EDO RAM, and the like. Memory 1506 may include other types of memory as well, or combinations thereof. Memory 1506 may be used to store one or more slates on a temporary basis, for example, in cache.

A user may interact with the computing device 1500 through a visual display device 1518, such as a screen or monitor, that may display one or more user interfaces 1520 that may be provided in accordance with exemplary embodiments. The visual display device 1518 may also display other aspects, elements and/or information or data associated with exemplary embodiments, e.g., visualizations of topographic image information. In exemplary embodiments, the visual display device 1518 may be a three-dimensional display. The computing device 1500 may include other I/O devices for receiving input from a user, for example, a keyboard or any suitable multi-point touch interface 1508, a pointing device 1510 (e.g., a mouse, a user's finger interfacing directly with a display device, etc.). The keyboard 1508 and the pointing device 1510 may be coupled to the visual display device 1518. The computing device 1500 may include other suitable conventional I/O peripherals.

The computing device 1500 may include one or more audio input devices 1524, such as one or more microphones, that may be used by a user to provide one or more audio input streams.

The computing device 1500 may include one or more storage devices 1524, such as a durable disk storage (which may include any suitable optical or magnetic durable storage device, e.g., RAM, ROM, Flash, USB drive, or other semiconductor-based storage medium), a hard-drive, CD-ROM, or other non-transitory computer readable media, for storing data and computer-readable instructions and/or software that implement exemplary embodiments as taught herein. The storage device 1524 may be provided on the computing device 1500 or provided separately or remotely from the computing device 1500. The storage device 1524 may be used to store computer readable instructions for implementing one or more methods/algorithms as described herein. Exemplary methods/algorithms descried herein may be programmatically implemented by a computer process in any suitable programming language, for example, a scripting programming language, an object-oriented programming language (e.g., Java), and the like. Thus, in exemplary embodiment the processor may be configured to process endoscopic image data relating to a plurality of illumination conditions to calculate topographic information for a sample, implement virtual chromoendoscopy, e.g., based on the calculated topographic information, and/or implement CAD of features such as leassions, e.g., based on the based on the calculated topographic information.

The computing device 1500 may include a network interface 1512 configured to interface via one or more network devices 1522 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1512 may include a built-in network adapter, network interface card, PCMCIA network card, card bus network adapter, wireless network adapter, USB network adapter, modem or any other device suitable for interfacing the computing device 1500 to any type of network capable of communication and performing the operations described herein. The network device 1522 may include one or more suitable devices for receiving and transmitting communications over the network including, but not limited to, one or more receivers, one or more transmitters, one or more transceivers, one or more antennae, and the like.

The computing device 1500 may run any operating system 1516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 1516 may be run in native mode or emulated mode. In an exemplary embodiment, the operating system 1516 may be run on one or more cloud machine instances.

In some embodiments, the computing device 1500 may implement a gesture recognition interface (for example, Kinnect/LEAP sensor type interface). In other embodiments, the computing device may interface with a control system placed in the handle of an endoscope. Such I/O implementations may be used to control the viewing angle of a 3D visualization of the topology associated with the image the endoscopist is reviewing. Thus, instead of physically changing the viewing angle on the image by means of moving the tip of the endoscope with respect to the object inspected, the practitioner could move the virtual representation of the topography.

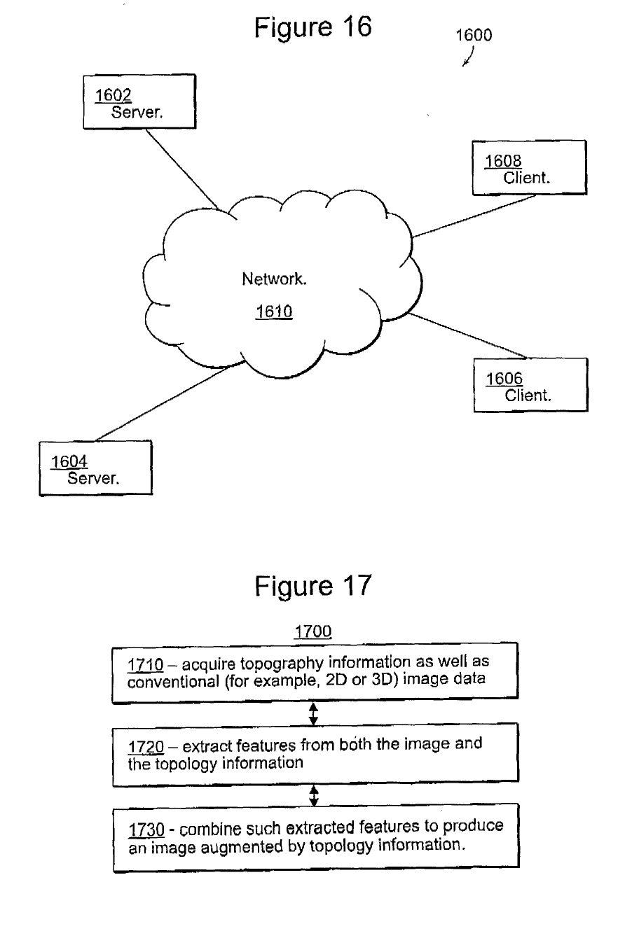

FIG. 16 depicts an exemplary network environment 1600 suitable for a distributed implementation of exemplary embodiments. The network environment 1600 may include one or more servers 1602 and 1604 coupled to one or more clients 1606 and 1608 via a communication network 1610. The network interface 1512 and the network device 1522 of the computing device 1500 enable the servers 1602 and 1604 to communicate with the clients 1606 and 1608 via the communication network 1610. The communication network 1610 may include, but is not limited to, the Internet, an intranet, a LAN (Local Area Network), a WAN (Wide Area Network), a MAN (Metropolitan Area Network), a wireless network, an optical network, and the like. The communication facilities provided by the communication network 1610 are capable of supporting distributed implementations of exemplary embodiments.

Although the teachings herein have been described with reference to exemplary embodiments and implementations thereof, the disclosed systems, methods and non-transitory storage medium are not limited to such exemplary embodiments/implementations. Rather, as will be readily apparent to persons skilled in the art from the description taught herein, the disclosed systems and methods are susceptible to modifications, alterations and enhancements without departing from the spirit or scope hereof. Accordingly, all such modifications, alterations and enhancements within the scope hereof are encompassed herein.

1. A photometric imaging endoscope system comprising:
an imaging endoscope device including one or more light sources and one or more detectors adapted for imaging a surface under each of the plurality of illumination conditions; and
a processor operatively associated with the imaging device and configured to calculate surface image data for the surface that is imaged under the plurality of illumination conditions and computing a high frequency spatial component of the calculated image data.

2. The system of claim 1, wherein the imaging device includes one or more light sources adapted for illuminating the surface from each of a plurality of imaging directions and a detector adapted for imaging the surface under illumination from each of the plurality of illumination directions and wherein the calculated surface image information includes a calculated surface normal map for the surface.

3. (canceled)

4. The system of claim 1, wherein the processor computes the high frequency spatial component of calculated topographical image information by filtering out low frequency spatial components of the calculated topographical image information

5. The system of claim 4, wherein the filtering out the low frequency spatial components of the calculated surface normal map includes calculating directional gradients for the surface by scaling the direction normal to the surface and high-pass filtering each of the directional gradients and wherein the high-pass filtering each of the directional gradients includes calculating a low frequency component as a convolution of the gradient with a Gaussian kernel and subtracting out the low frequency component and wherein the processor is further configured to calculate a height map of the surface by integrating the filtered gradients.

6. (canceled)

7. (canceled)

8. The system of claim 1, wherein the imaging device is characterized by significant variation of light source directional vectors across a field of view resulting from at least one of (i) a wide field of view and (ii) small working distance illumination, wherein the significant variation of the light source directional vectors manifests as low-spatial frequency artifacts for calculating topographical image information.

9. The system of claim 1, wherein the imaging endoscope comprises a tubular body with a plurality of distal light emitters such that the imaging endoscope comprises a plurality of at least three light sources.

10. (canceled)

11. The system of claim 10 wherein the light sources are connected to a controller from 12 that is operative to actuate the plurality of light sources in a temporal sequence to obtain a plurality of images.

12. (canceled)

13. The system of claim 1 wherein the endoscope comprises a handle connected to a tubular endoscope body, the handle having a control panel that actuates an imaging procedure and includes a data processor that processes image data and a memory that stores image data.

14. The system of claim 1 wherein the endoscope comprises a colonoscope.

15. (canceled)

16. (canceled)

17. (canceled)

18. (canceled)

19. (canceled)

20. The system of claim 2, wherein the surface normal map is calculated in a calibrated domain.

21. The system of claim 1, wherein the processor is further configured to at least one of (i) register images and (ii) translate images acquired by the detector in order to account for relative motion between the imaging device and target.

22. The system of claim 1, wherein the detector is interlaced and wherein the processor is further configured to extract data related to two different illumination conditions from each frame acquired by the detector.

23. The system of claim 1, wherein the imaging of the surface includes high dynamic range imaging of the surface by changing at least one of (i) an intensity of illumination and (ii) a sensitivity of detection.

24. The system of claim 1, further comprising a display for displaying a virtual image of the surface derived from the filtered surface normal map of the surface.

25. The system of claim 1 wherein calculated topographical image information includes a calculation of surface orientation of each pixel in a field of view and wherein the surface orientation is represented by at least one of (i) a surface normal, (ii) a surface parallel vector, or (iii) are equation of a plane and wherein the surface orientations are reconstructed into a surface topography.

26. (canceled)

27. (canceled)

28. The system of claim 1 wherein the imaging endoscope device comprises a plurality of light sources that are symmetrically positioned relative to a detector.

29. The system of claim 1 wherein calculated topographical image information is used to reconstruct information relating to features in a surface a curved complex geometry or in a surface with heterogeneous optical properties.

30. The system of claim 1 wherein the imaging device is adapted to simultaneously acquire both the topographical image information and two-dimensional image information.

31. The system of claim 1 wherein the processor is configured to overlay the topographical image information with respect to the two-dimensional image information and is configured to implement virtual chromoendoscopy based at least in part on topographical image information and further configured to implement computer aided diagnosis/detection (CAD) of one or more features based at least in part on topographical image information.

32. (canceled)

33. (canceled)

34. The system of claim 1 further comprising illumination fiber optics operatively associated with at the one or more light sources adapted for illuminating the surface and imaging fiber optics operatively associated with the one or more detectors adapted for receiving light from the surface.

35. (canceled)

36. The system of claim 1 wherein the one or more light sources are adapted to provide diffuse illumination across a wide field of view greater than 90 degrees and the one or more light sources are operatively associated with at least one of a diffuser element or a cross polarizer.

37. (canceled)

38. The system of claim 1 wherein the one or more light detectors are adapted to at least one of reduce specular reflection or enhance contrast and saturation.

39. The system of claim 1 wherein the one or more light detectors are operatively associated with a cross polarizer.

40. The system of claim 1 wherein the imaging device includes a plurality of light sources and a single detector and the plurality of light sources wherein source separation is less than 14 mm.

41. The system of claim 1 wherein the imaging device includes a single light source and a plurality of detectors.

42. The system of claim 1 wherein at least one of the one or more light sources and at least one of the one or more detectors are movable relative to one another.

43. The system of claim 1 wherein the plurality of illumination conditions are each characterized by a common field of view and the processor is configured to index images acquired for each of the plurality of illumination conditions.

44. (canceled)

45. (canceled)

46. The system of claim 1 wherein topographical image information is sufficient to resolve a surface feature less than 1 mm in height or depth at working distances of 10-40 mm.

47. The system of claim 1 wherein the one or more light sources include white light sources.

48. The system of claim 1 wherein the one or more light sources emit light with different spectral bands.

49. The system of claim 1 wherein sequential illumination by a plurality of light sources is synchronized to a detection frame rate.

50. The system of claim 1 wherein each of the one or more light sources is operatively associated with a holographic light shaping diffuser or the one or more light sources is operatively associated with a linear polarizer in a cross-configuration.

51. (canceled)

52. The system of claim 1 wherein image data acquired by the one or more detectors is processed using a de-mosaicing interpolation process implemented by the processor to provide full resolution RGB images from Bayer-patterned images.

53. The system of claim 1 wherein calculating the topographical information includes using an approximation or the light remitted from the sample according to Lambertian reflectance.

54. The system of claim 1 wherein calculating the topographical information includes using an approximation or the light remitted from the sample according to a Phong model or another model that accounts for shadowing and specular reflections.

55. The system of claim 5, wherein the filtered gradients are integrated using a multigrid solver for the Poisson equation that reduces integration inconsistency errors.

56. The system of claim 1 wherein the endoscope device comprises a tubular body having an array of light sources to emit light from a plurality of regions on an outer surface of the tubular body and the endoscope device has one or more light detectors on an outer surface of the tubular body.

57. (canceled)

58. The system of claim 1 wherein the endoscope device has a plurality of light sources that illuminate a plurality of regions on the surface wherein an illumination region of a first light source overlays an illumination region of a second light source and the first light source is positioned on an outer sidewall of the endoscope and the second light source is positioned on a distal surface of the endoscope and wherein overlapping illumination region is on an inner surface of a body lumen.

59. (canceled)

60. (canceled)

61. The system of claim 1 wherein the endoscope device comprises a capsule, the capsule comprises a housing shaped to be orally administered to a patient and the capsule comprises a batter, a memory, and a wireless transmitter.

62. (canceled)

63. (canceled)

64. The system of claim 61 wherein the capsule has a plurality of LED light sources or laser diodes and a detector.

65. (canceled)

66. (canceled)

67. (canceled)

68. (canceled)

69. (canceled)

70. A method of photometric imaging comprising:
an illuminating a surface with light from one or more light sources to image the surface under a plurality of illumination conditions;
detecting light form the surface with one or more detectors; and
processing image data from the one or more detectors with a data processor that is configured to calculate surface image data for the surface based on imaging of the surface under the plurality of illumination conditions and computing a high frequency spatial component of the calculated surface image data.

71. The method of claim 70 further comprising illuminating the surface from each of a plurality of imaging directions and a detector adapted for imaging the surface under illumination from each of the plurality of illumination directions.

72. The method of claim 70 further comprising calculating a surface normal map of the surface and computing the high frequency spatial component of the calculated surface image data by filtering out low frequency spatial components of the calculated surface image data and filtering out the low frequency spatial components of the calculated surface normal map by calculating directional gradients for the surface, scaling the direction normal to the surface and high-pass filtering each of the directional gradients and high-pass filtering each of the directional gradients by calculating a low frequency component as a convolution of the gradient with a Gaussian kernel and subtracting out the low frequency component and calculating a height map of the target surface by integrating the filtered gradients.

73. (canceled)

74. (canceled)

75. (canceled)

76. (canceled)

77. The method of claim 70 further comprising controlling actuators of a plurality of light source directional vectors across a field of view resulting from at least one of (i) a wide field of view and (ii) small working distance illumination, wherein the significant variation of the light source directional vectors manifests as low-spatial frequency artifacts when calculating the topographical image information.

78. (canceled)

79. The method of claim 70 further comprising actuating a plurality of light sources at a distal end of the endoscope in sequence and imaging the surface with an endoscope.

80. (canceled)

81. (canceled)

82. The method of claim 70 further comprising actuating control elements of a control panel on an endoscope handle and further comprising transmitting images from the handle to an external storage device and processing image data with a data processor in the handle.

83. (canceled)

84. (canceled)

85. The method of claim 72 further comprising calculating the surface normal map in a calibrated domain.

86. The method of claim 70 further comprising processing the images to at least one of (i) register images and (ii) translate images acquired by the detector in order to account for relative motion between the imaging device and the surface.

87. The method of claim 70 further comprising interlacing the detector and extracting data related to two different illumination conditions from each frame acquired by the detector.

88. The method of claim 70 further comprising high dynamic range imaging of a target surface by changing at least on of (i) an intensity of illumination and (ii) a sensitivity of detection.

89. The method of claim 70 further comprising displaying a virtual image of the surface derived from the filtered surface normal map of the surface.

90. The method of claim 70 further comprising calculating of surface orientation of each detector pixel in a field of view and representing the surface orientation by at least one of (i) a surface normal, (ii) a surface parallel vector, or (iii) an equation of a plane and reconstructing the surface orientations into a surface topography.

91. (canceled)

92. (canceled)

93. (canceled)

94. The method of claim 70 further comprising calculating topographical image information to reconstruct information relating to features in a target surface with a complex geometry or in a target surface with heterogeneous optical properties and acquiring both the topographical image information and two-dimensional image information and overlaying the topographical image information with respect to the two-dimensional image information.

95. (canceled)

96. (canceled)

97. The method of claim 70 further comprising performing virtual chromoendoscopy based at least in part on processed topographical image information.

98. The method of claim 70 further comprising performing computer aided diagnosis/detection (CAD) of one or more features based at least in part on processed topographical image information.

99. The method of claim 70 further comprising optically coupling the one or more light sources to illuminate the surface with a plurality of optical fibers and optically coupling one or more detectors adapted for receiving light from the surface with optical fibers.

100. (canceled)

101. The method of claim 70 further comprising diffusely illuminating a wide field of view on the surface.

102. The method of claim 70 wherein the one or more light sources are operatively associated with at least one of a diffuser element or a cross polarizer.

103. The method of claim 70 wherein the one or more light detectors are adapted to at least one of reduce specular reflection or enhance contrast and saturation.

104. (canceled)

105. The method of claim 70 further comprising detecting light with the imaging device that includes a plurality of light sources and a single detector.

106. The method of claim 70 further comprising detecting light with the imaging device includes a single light source and a plurality of detectors.

107. The method of claim 70 further comprising providing relative movement between at least one of the one or more light sources and at least one of the one or more detectors.

108. The method of claim 70 wherein the plurality of illumination conditions are each characterized by a common field of view.

109. The method of claim 70 further comprising indexing images acquired for each of the plurality of illumination conditions.

110. The method of claim 70 further comprising operating the imaging device that includes a plurality of light sources wherein the light source separation is greater than 1 mm and less than 14 mm.

111. The method of claim 70 further comprising determining topographical image information sufficient to resolve feature less than 1 mm in height or depth at working distances of 10-40 mm.

112. The method of claim 70 wherein the one or more light sources include white light sources, or wherein the one or more light sources include spectrum band specific light sources.

113. (canceled)

114. The method of claim 70 further comprising sequentially illuminating a surface with a plurality of light sources that are synchronized to a detection frame rate and each of the one or more light sources is operatively associated with a holographic light shaping diffuser or each of the one or more light sources is operatively associated with a linear polarizer in a cross-configuration.

115. (canceled)

116. (canceled)

117. The method of claim 70 further comprising generating data by the one or more detectors that is processed using a de-mosaicing interpolation process implemented by the processor to provide full resolution RGB images from Bayer-patterned images.

118. The method of claim 70 further comprising calculating topographical information including using an approximation or the light remitted from the surface according to Lambertian reflectance and calculating topographical information including using an approximation or the light remitted from the sample according to a Phong model or another model that accounts for shadowing and specular reflections.

119. (canceled)

120. The method of claim 72 further comprising processing the filtered gradients using a multigrid solver for the Poisson equation that reduces integration inconsistency errors.

121. The method of claim 70 further comprising generating high frequency image data and low frequency image data and processing the high frequency image data and generating a composite image with the processed high frequency image data and the low frequency image data.

122. (canceled)

123. The method of claim 79 further comprising delivering a distal end of the endoscope into a lumen of a patient.

124. The method of claim 79 further comprises orally administering and endoscope capsule to a patient wherein the capsule comprise at least two light sources, a battery, a detector and a transmitter.

125. (canceled)

126. The method of claim 70 illuminating a region of interest from different direction to generate quantitative image data.

127. (canceled)

128. The method of claim 70 further comprising gating imaging times to correlate with the plurality of illumination directions.

129. The method of claim 70 further comprising imaging using a plurality of illumination conditions including a plurality of focal locations, a plurality of speckle patterns or a plurality of different phases.

130. A photometric stereo imaging system for high frequency topography comprising:
an imaging device including one or more light sources and one or more detectors adapted for imaging a target surface under each of the plurality of illumination conditions; and
a processor operatively associated with the imaging device and configured to calculate topographical image information for the target surface based on imaging of the target surface under the plurality of illumination conditions and computing a high frequency spatial component of the calculated topographical image information.

131. The system of claim 130, wherein the imaging device includes one or more light sources adapted for illuminating the target surface from each of a plurality of imaging directions and a detector adapted for imaging the target surface under illumination from each of the plurality of illumination directions.

132. The system of claim 130 wherein the calculated topographical image information includes a calculated surface normal map for the target surface.

133. The system of claim 130, wherein the processor computes the high frequency spatial component of the calculated topographical image information by filtering out low frequency spatial components of the calculated topographical image information, the processor further configured to calculate a height map of the target surface by integrating the filtered gradients.

134. The system of claim 133, wherein the filtering out the low frequency spatial components of the calculated surface normal map includes calculating directional gradients for the target surface by scaling the direction normal to the surface and high-pass filtering each of the directional gradients and wherein the high-pass filtering each of the directional gradients includes calculating a low frequency component as a convolution of the gradient with a Gaussian kernel and subtracting out the low frequency component.

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. A computer assisted detection (CAD) system for characterizing a physical feature in a body cavity, the system comprising:
an imaging device including one or more light sources and one or more detectors adapted for imaging a surface at each of the plurality of illumination conditions or from different viewing directions;
a processor operatively associated with the imaging device and configured to image a body cavity surface under the plurality of illumination conditions or different viewing directions to determine a characteristic of a physical feature in the body cavity based on a combination of one or more imaging parameters.

151. The CAD system of claim 150, wherein the imaging device is an endoscope and wherein the physical feature is a polyp, a lesion or other abnormality.

152. (canceled)

153. The CAD system of claim 150, wherein the one or more parameters relating to the individual image are selected from the group consisting of: (i) color, (ii) contrast, (iii) vesselness and (iv) Sobel edges and wherein the one or more parameters relating to the calculated topographical information are selected from the group consisting of: (i) curvature, (ii) orientation of a surface normal and (iii) divergence of a surface normal.

154. (canceled)

155. The CAD system of claim 150, wherein the processor applies a machine learned algorithm for characterizing the physical feature.

156. The CAD system of claim 150, wherein one or more features identified by the CAD process are indicated on an image composed of some combination of the detected images by an arrow, a marker, or contrast enhancement.

157. The CAD system of claim 150 further comprising a nontransitory computer readable medium having stored thereon a sequence of instruction to compute a characteristic from detected image data.

158. The CAD system of claim 150 wherein the processor is configured to calibrate an illumination system or wherein the processor is configured to calibrate a detector system.

159. (canceled)

160. The system of claim 157 further comprises computing an image using detector distortion parameters.

161. (canceled)

162. (canceled)

163. (canceled)

164. (canceled)

165. (canceled)

166. (canceled)

167. (canceled)

168. (canceled)

169. (canceled)

170. A method for characterizing a physical feature in a body cavity, the method comprising:
imaging a target surface under each of a plurality of illumination conditions or from different viewing directions;
calculating topographical image information for the surface based on the imaging of the surface under the plurality of illumination conditions or different viewing directions; and
characterizing a physical feature in the body cavity based on a combination of one or more parameters relating to an individual image and one or more parameters relating to the calculated topographical imaging information.

171. The method of claim 170 further comprising determining a texture of a tissue surface or determining a surface topology of the tissue.

172. The method of claim 170 further comprising measuring a quantitative characteristic of a region of tissue.

173. The method of claim 172 further comprising phase imaging the tissue.

174. (canceled)

175. The method of claim 170 further comprising illuminating the tissue with light from a plurality of directions.

176. (canceled)

177. (canceled)

178. (canceled)

179. (canceled)

180. A computer assisted detection (CAD) system for characterizing a physical feature in a body cavity, the system comprising:
an imaging device including one or more light sources and one or more detectors adapted for imaging a surface under each of the plurality of illumination conditions or different viewing directions;
a processor operatively associated with the imaging device and configured to (i) calculate topographical image information for the target surface based on the imaging of the target surface under the plurality of illumination conditions; and (ii) overlay the calculated topographical imaging information with respect to an individual image.

181. The system of claim 180 wherein the processor is connected to a memory for storing images.

182. The system of claim 180 wherein the processor is configured to execute instructions stored on a non-transitory computer readable medium to calculate the image information.

183. (canceled)

184. (canceled)

185. (canceled)

186. (canceled)

187. (canceled)

188. (canceled)

189. (canceled)

190. A method for characterizing a physical feature in a body cavity the method comprising:
imaging a surface under each of the plurality of illumination conditions;
calculating topographical image information for the surface based on the imaging of the surface under the plurality of illumination conditions or different points of view; and
overlaying the calculated topographical imaging information with respect to a detected image.

191. (canceled)

192. (canceled)

193. (canceled)

194. The method of claim 190 wherein the imaging step comprises actuating a plurality of light sources to illuminate a tissue surface and wherein the actuating step comprises operating a light source controller.

195. (canceled)

196. The method of claim 190 wherein the overlaying step comprises altering a color and intensity of a plurality of image pixels.

197. The method of claim 190 wherein the overlaying step denotes a region of mucosal tissue or wherein the overlaying step denotes a cancerous lesion.

198. (canceled)

199. (canceled)

200. (canceled)
